(19) United States
(12) Patent Application Publication
(10) Pub. No.: US 2024/0165082 A1
(43) Pub. Date: May 23, 2024

(54) ORALLY ADMINISTERED COMPOSITIONS FOR CANCER TREATMENT

(71) Applicant: Alberto Paz, Indian Rocks Beach, FL (US)

(72) Inventor: Alberto Paz, Indian Rocks Beach, FL (US)

(21) Appl. No.: 18/496,730

(22) Filed: Oct. 27, 2023

Related U.S. Application Data
(63) Continuation of application No. PCT/US2022/029188, filed on May 13, 2022.
(60) Provisional application No. 63/190,409, filed on May 19, 2021.

(51) Int. Cl. A61K 31/4172; A61K 9/16; A61K 9/20; A61K 9/28; A61P 35/00

(52) U.S. Cl. CPC A61K 31/4172; A61K 9/1617; A61K 9/1635; A61K 9/1682; A61K 9/2013; A61K 9/284; A61P 35/00

(57) ABSTRACT

Disclosed herein is a pharmaceutical composition comprising delayed release beads that include a solid dispersion of at least one histamine salt and a delayed release coating. The composition provides a therapeutically effective plasma concentration of at least about 0.2 μmole/L. Also disclosed herein is a method for treating a cancer or a tumor including administering the pharmaceutical composition to a subject having the cancer or the tumor.

### CROSS-REFERENCE TO RELATED APPLICATIONS

This application is a continuation of PCT Application No. PCT/US2022/029188, filed May 13, 2022, which claims priority to and the benefit of U.S. Provisional Patent Application No. 63/190,409, filed May 19, 2021, each of which are incorporated herein by reference. Any and all applications for which a foreign or domestic priority claim is identified in the Application Data Sheet as filed with the present application are hereby incorporated by reference under 37 C.F.R. § 1.57.

### BACKGROUND

### Field of the Disclosure

The present disclosure relates generally to compositions of histamine and histamine receptor agonists with delayed release capability, and methods of making and using such compositions. The compositions comprise solid dispersions of histamine and histamine receptor agonists and a delayed release coating. In particular, the compositions can be orally administered as a method for the treatment of solid tumors and leukemias.

### Description of the Related Art

Histamine regulates cancer-associated biological processes during cancer development in multiple cell types, including neoplastic cells and cells in the tumor micro-environment. Treatment outcomes depend on tumor cell type, the level of expression of histamine receptors, signal transduction associated with these receptors, tumor micro-environment and histamine metabolism, reinforcing the complexity of cancer disease. The pivotal role of histamine receptors in the development and progression of many types of cancers indicates that the receptors are a promising molecular therapeutic target for cancer treatment. Some histamine receptors are differentially expressed in tumors compared with normal tissues, and this expression is associated with clinicopathological characteristics in most cancer types, suggesting that histamine receptors might represent a novel cancer biomarker.

Oral administration of a pharmaceutical composition containing histamine is complicated by the gastro-intestinal (GI) tract. For example, while an orally administered pharmaceutical composition passes through the human digestive tract, it is subjected to pH in stomach between 1.2 and 4.0, which has potential to inactivate histamine. Additionally, histamine can stimulate the histamine receptors of parietal cells in the stomach and trigger the release of additional acid in the stomach. As a result, oral administration of a pharmaceutical composition containing histamine by existing methods can be very harmful to the human GI tract.

The need exists for a GI-safe orally administered pharmaceutical composition containing histamine for the treatment of solid tumors and leukemias.

### SUMMARY

Disclosed herein is a pharmaceutical composition comprising delayed release beads. In several embodiments, the delayed release beads include a solid dispersion of a therapeutically effective dosage of at least one active pharmaceutical ingredient (API). In several embodiments, the API comprises histamine, a histamine salt, a histamine derivative, a salt of a histamine derivative, and any combination of the foregoing. In several embodiments, the delayed release coating includes a water insoluble polymer and an enteric polymer. In several embodiments, the delayed release coating is insoluble at pH equal to or less than about 3.5. In several embodiments, the delayed release coating is soluble at pH equal to or greater than about 6. In several embodiments, the composition provides a therapeutically effective plasma concentration of the API of equal to or greater than about 0.2 μmole/L. In several embodiments, the composition provides the therapeutically effective plasma concentration for about 0.25 to about 5 hours.

In several embodiments, the histamine salt comprises a histamine monocation or a histamine polycation. In several embodiments, the histamine salt comprises at least one anion selected from acetate, aspartate, citrate, formate, fumarate, halide, malate, nitrate, nitrite, phosphite, phosphate, succinate, sulfate, sulfite, tartrate, and any combination of the foregoing. In several embodiments, the histamine salt comprises histamine dihydrochloride. In several embodiments, the histamine derivative comprises a C₁₋₆ alkyl histamine. In several embodiments, the histamine derivative comprises N-methylhistamine or 4-methylhistamine. In several embodiments, the salt of the histamine derivative comprises a C₁₋₆ alkyl histamine monocation or a C₁₋₆ alkyl histamine polycation. In several embodiments, the salt of the histamine derivative comprises at least one anion selected from acetate, aspartate, citrate, formate, fumarate, halide, malate, nitrate, nitrite, phosphite, phosphate, succinate, sulfate, sulfite, tartrate, and any combination of the foregoing. In several embodiments, the salt of the histamine derivative comprises N-methylhistamine dihydrochloride or 4-methylhistamine dihydrochloride.

In several embodiments, the solid dispersion comprises at least one a pharmaceutically acceptable solid buffer comprising an organic acid or an alkaline buffer. In several embodiments, the organic acid comprises aspartic acid, fumaric acid, acetic acid, formic acid, succinic acid, lactic acid, malic acid, tartaric acid, citric acid, ascorbic acid, nicotinic acid, methanesulfonic acid, ethanesulfonic acid, p-toluensulfonic acid, salicylic acid, naphthalenesulfonic acid, and any combination of the foregoing. In several embodiments, the alkaline buffer comprises a cation comprising an alkali metal, an alkaline earth metal and any combination of the foregoing and an anion comprising acetate, citrate, formate, halide, malate, nitrate, nitrite, phosphite, phosphate, succinate, sulfate, sulfite, tartrate, and any combination of the foregoing.

In several embodiments, the API binds histamine H2 receptors selectively. In several embodiments, the API inhibits the production or release of intracellular hydrogen peroxide.

In several embodiments, the API has a solubility equal to or greater than about 94%. In several embodiments, the solubility is measured at a pH of about of 7-7.8.

In several embodiments, the API has an apparent permeability across the small intestine equal to or greater than about 18 microcentimeters per second (μcm/s). In several embodiments, the apparent permeability is measured at a pH of about of 7-7.8. In several embodiments, the API is absorbed in the small intestine in an amount equal to or greater than 85% of the orally administered dose. In several embodiments, the therapeutically effective dosage is from about 0.1 mg to about 10 mg. In some embodiments, the pharmaceutical composition is for use in treating a cancer or a tumor. In some embodiments, the pharmaceutical composition is for use in treating a malignant growth. In some embodiments, the pharmaceutical composition is for use in inhibiting replication of a malignant growth or a tumor. In some embodiments, the pharmaceutical composition is for treating a cancer or a tumor. In some embodiments, the pharmaceutical composition is for treating a malignant growth. In some embodiments, the pharmaceutical composition is for inhibiting replication of a malignant growth or a tumor. In some embodiments, the pharmaceutical composition is formulated as an oral dosage. In some embodiments, an aspect of the present disclosure is the use of an effective amount of a pharmaceutical composition disclosed herein in the manufacture of a medicament for treating a cancer or a tumor. In some embodiments, the use of an effective amount of a pharmaceutical composition is in the manufacture of a medicament for treating a malignant growth or a tumor. In some embodiments, the use of an effective amount of a pharmaceutical composition is in the manufacture of a medicament for inhibiting replication of a malignant growth or a tumor. In some embodiments, the use is for a pharmaceutical composition formulated as an oral dosage.

In another aspect, a pharmaceutical composition comprising an enteric tablet is disclosed. The enteric tablet includes a compressed tablet having an outer surface and a polymeric coating located on the outer surface, wherein the compressed tablet comprises a therapeutically effective dosage of at least one API, wherein the API comprises histamine, a histamine salt, a histamine derivative, a salt of a histamine derivative, and any combination of the foregoing, and wherein the polymeric coating is insoluble at pH equal to or less than about 3.5 and is soluble at pH equal to or greater than about 6.

In some embodiments, the composition provides a therapeutically effective plasma concentration of the API of equal to or greater than about 0.2 μmole/L. In some embodiments, the composition provides the therapeutically effective plasma concentration for about 0.25 to about 5 hours. In some embodiments, the histamine salt comprises a histamine monocation or a histamine polycation. In some embodiments, the histamine salt comprises at least one anion selected from acetate, aspartate, citrate, formate, fumarate, halide, malate, nitrate, nitrite, phosphite, phosphate, succinate, sulfate, sulfite, tartrate, and any combination of the foregoing. In some embodiments, the histamine salt comprises histamine dihydrochloride. In some embodiments, the histamine derivative comprises a C1-6 alkyl histamine. In some embodiments, the histamine derivative comprises N-methylhistamine, 1-methylhistamine, 2-methylhistamine, 4-methylhistamine, 5-methylhistamine, alpha-methylhistamine, and any combination of the foregoing. In some embodiments, the salt of the histamine derivative comprises a C1-6 alkyl histamine monocation or a C1-6 alkyl histamine polycation. In some embodiments, the salt of the histamine derivative comprises at least one anion selected from acetate, aspartate, citrate, formate, fumarate, halide, malate, nitrate, nitrite, phosphite, phosphate, succinate, sulfate, sulfite, tartrate, and any combination of the foregoing. In some embodiments, the salt of the histamine derivative comprises N-methylhistamine dihydrochloride, 1-methylhistamine dihydrochloride, 2-methylhistamine dihydrochloride, 4-methylhistamine dihydrochloride, 5-methylhistamine dihydrochloride, alpha-methylhistamine dihydrochloride, and any combination of the foregoing. In some embodiments, the solid dispersion comprises at least one a pharmaceutically acceptable solid buffer comprising an organic acid or an alkaline buffer. In some embodiments, the organic acid comprises aspartic acid, fumaric acid, acetic acid, formic acid, succinic acid, lactic acid, malic acid, tartaric acid, citric acid, ascorbic acid, nicotinic acid, methanesulfonic acid, ethanesulfonic acid, p-toluensulfonic acid, salicylic acid, naphthalenesulfonic acid, and any combination of the foregoing. In some embodiments, the alkaline buffer comprises a cation comprising an alkali metal, an alkaline earth metal and any combination of the foregoing and an anion comprising acetate, citrate, formate, halide, malate, nitrate, nitrite, phosphite, phosphate, succinate, sulfate, sulfite, tartrate, and any combination of the foregoing. In some embodiments, the API binds histamine H2 receptors selectively. In some embodiments, the API inhibits the production or release of intracellular hydrogen peroxide. In some embodiments, the API has a solubility equal to or greater than about 94%. In some embodiments, the solubility is measured at a pH of about of 7-7.8. In some embodiments, the API has an apparent permeability across the small intestine equal to or greater than about 18 microcentimeters per second (μcm/s). In some embodiments, the wherein the apparent permeability is measured at a pH of about of 7-7.8. In some embodiments, the API is absorbed in the small intestine in an amount equal to or greater than 85% of the therapeutically effective dosage. In some embodiments, the therapeutically effective dosage is from about 0.1 mg to about 10 mg. In some embodiments, the composition is for use in treating a cancer or a tumor. In some embodiments, the composition is for use in the manufacture of a medicament for treating a cancer or a tumor. In some embodiments, the composition is formulated as an oral dosage.

Disclosed herein is a method of preparing the delayed release beads. In several embodiments, the method includes dissolving the API and sufficient solubility-enhancing polymer in a pharmaceutically acceptable solvent, to form a solution. In several embodiments, the method includes removing the pharmaceutically acceptable solvent from the solution, whereby particles of a solid dispersion are formed. In several embodiments, the method includes dissolving the water insoluble polymer and the enteric polymer in a pharmaceutically acceptable coating solvent, thereby forming a delayed release coating solution. In several embodiments, the method includes coating the particles of solid dispersion with the delayed release coating solution. In several embodiments, the method includes removing the coating solvent, thereby forming delayed release beads, the beads including a delayed release coating formed on the particles of the solid dispersion.

Disclosed herein is a method for treating a cancer or a tumor. In several embodiments, the method includes administering a pharmaceutical composition to a subject having the cancer or the tumor. In several embodiments, the pharmaceutical composition of the method includes delayed release beads. In several embodiments, the delayed release beads of the method include a solid dispersion of a therapeutically effective dosage of at least one active pharmaceutical ingredient (API). In several embodiments, the API of the method includes histamine, a histamine salt, a histamine derivative, a salt of a histamine derivative, and any combination of the foregoing. In several embodiments, the delayed release beads of the method include a delayed release coating that includes a water insoluble polymer and an enteric polymer. In several embodiments, the delayed release coating of the method is insoluble at pH equal to or less than about 3.5 and is soluble at pH equal to or greater than about 6.

In several embodiments, the method includes providing a therapeutically effective plasma concentration of the API of equal to or greater than about 0.2 μmole/L.

In several embodiments, the method includes providing the therapeutically effective plasma concentration for about 0.25 to about 5 hours. In some embodiments, the histamine salt comprises at least one anion selected from acetate, aspartate, citrate, formate, fumarate, halide, malate, nitrate, nitrite, phosphite, phosphate, succinate, sulfate, sulfite, tartrate, and any combination of the foregoing. In some embodiments, the histamine salt comprises histamine dihydrochloride. In some embodiments, the histamine derivative comprises a C1-6 alkyl histamine. In some embodiments, the histamine derivative comprises N-methylhistamine or 4-methylhistamine. In some embodiments, the salt of the histamine derivative comprises a C1-6 alkyl histamine monocation or a C1-6 alkyl histamine polycation. In some embodiments, the salt of the histamine derivative comprises at least one anion selected from acetate, aspartate, citrate, formate, fumarate, halide, malate, nitrate, nitrite, phosphite, phosphate, succinate, sulfate, sulfite, tartrate, and any combination of the foregoing. In some embodiments, the salt of the histamine derivative comprises N-methylhistamine dihydrochloride or 4-methylhistamine dihydrochloride. In some embodiments, the API inhibits the production or release of intracellular hydrogen peroxide. In some embodiments, the cancer is a leukemia.

In several embodiments of the method, the histamine salt comprises a histamine monocation or a histamine polycation. In several embodiments of the method, the histamine salt comprises at least one anion selected from acetate, aspartate, citrate, formate, fumarate, halide, malate, nitrate, nitrite, phosphite, phosphate, succinate, sulfate, sulfite, tartrate, and any combination of the foregoing. In several embodiments of the method, the histamine salt comprises histamine dihydrochloride. In several embodiments of the method, the histamine derivative comprises a C₁₋₆ alkyl histamine. In several embodiments, the histamine derivative comprises N-methylhistamine or 4-methylhistamine. In several embodiments of the method, the salt of the histamine derivative comprises a C₁₋₆ alkyl histamine monocation or a C₁₋₆ alkyl histamine polycation. In several embodiments of the method, the salt of the histamine derivative comprises at least one anion selected from acetate, aspartate, citrate, formate, fumarate, halide, malate, nitrate, nitrite, phosphite, phosphate, succinate, sulfate, sulfite, tartrate, and any combination of the foregoing. In several embodiments of the method, the salt of the histamine derivative comprises N-methylhistamine dihydrochloride or 4-methylhistamine dihydrochloride.

In several embodiments of the method, the API binds histamine H2 receptors selectively. In several embodiments of the method, the API inhibits the production or release of intracellular hydrogen peroxide.

In several embodiments of the method, the API has a solubility equal to or greater than about 94%. In several embodiments, the solubility is measured at a pH of about of 7-7.8.

In several embodiments of the method, the API has an apparent permeability across the small intestine equal to or greater than about 18 microcentimeters per second (μcm/s). In several embodiments, the apparent permeability is measured at a pH of about of 7-7.8.

In several embodiments of the method, the API is absorbed in the small intestine in an amount equal to or greater than 85% of the orally administered dose.

In several embodiments of the method, the therapeutically effective dosage is from about 0.1 mg to about 10 mg.

In several embodiments, the method further comprises administering the pharmaceutical composition orally.

In several embodiments, the method further comprises a lag time, the lag time is from about 0.25 h to about 3 h.

In several embodiments, the method avoids inactivation of natural killer cells in the tumor micro-environment. In several embodiments, the method increases activation of natural killer cells in the tumor micro-environment.

In several embodiments, the method further comprises administering at least one other cancer therapy. In several embodiments, the cancer therapy is selected from surgery, radiation, chemotherapy, immunotherapy, and any combination of the foregoing. In several embodiments, the cancer therapy comprises treatment with a cytokine. In several embodiments, the cancer therapy comprises treatment with Interleukin-2.

In several embodiments, cancer or the tumor is selected from a leukemia, a myeloma, a bone marrow cancer, a Hodgkin's lymphoma, a Non-Hodgkin's lymphoma, a follicular lymphoma, a lymphoid malignancy of T-cell or B-cell origin, a bladder cancer, a brain cancer, a breast cancer, a cervical cancer, a colorectal cancer, an esophageal cancer, a hepatocellular cancer, a melanoma, a head and neck cancer (including oral cancer), an ovarian cancer, a small cell lung cancer, a non-small cell lung cancer, a prostate cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma, a sarcoma, a carcinoma, an Ewings's tumor and a Wilm's tumor.

In several embodiments, the cancer is a leukemia. In several embodiments, the cancer is a leukemia selected from an acute myeloid leukemia, a lymphoblastic leukemia, a myelogenous leukemia, a chronic lymphocytic leukemia, a chronic myeloid leukemia, and any combination of the foregoing.

Disclosed herein is a method for treating a cancer comprising contacting a malignant growth or a tumor with the pharmaceutical composition. Further disclosed herein is a method for inhibiting replication of a malignant growth or a tumor comprising contacting the growth or the tumor with the pharmaceutical composition. Also disclosed herein is a use of an effective amount of the pharmaceutical composition in the manufacture of a medicament for treating a cancer or a tumor.

Disclosed herein is the pharmaceutical composition for use in treating a cancer or a tumor. Some embodiments relate to the pharmaceutical composition for use in treating a malignant growth. Some embodiments relate to the pharmaceutical composition for use in inhibiting replication of a malignant growth or a tumor. In several embodiments, the pharmaceutical composition is formulated as an oral dosage.

Disclosed herein is the use of the pharmaceutical composition for treating a cancer or a tumor. Some embodiments relate to the use of the pharmaceutical composition for treating a malignant growth. Some embodiments relate to the use of the pharmaceutical composition for inhibiting replication of a malignant growth or a tumor. In several embodiments, the pharmaceutical composition is formulated as an oral dosage.

Disclosed herein is the use of an effective amount of the pharmaceutical composition in the manufacture of a medicament for treating a malignant growth or a tumor. Further disclosed herein is a use of an effective amount of the pharmaceutical composition in the manufacture of a medicament for inhibiting replication of a malignant growth or a tumor. In several embodiments, the pharmaceutical composition is formulated as an oral dosage.

### BRIEF DESCRIPTION OF THE DRAWINGS

FIG. 1 illustrates a cross-section of one embodiment of a delayed release bead in accordance with an embodiment of the present disclosure.

FIG. 2 illustrates a cross-section of one embodiment of an enteric tablet in accordance with an embodiment of the present disclosure.

FIG. 3A illustrates the coefficient of correlation for the relationship between human absorption data and apparent permeability for MATTEK EPIINTESTINAL SMI microtissues.

FIG. 3B illustrates the coefficient of correlation for the relationship between human absorption data and apparent permeability for Caco-2 monolayer cells.

FIG. 4A illustrates the cumulative receiver concentration over time for an active pharmaceutical ingredient in accordance with an embodiment of the present disclosure.

FIG. 4B illustrates the cumulative receiver concentration over time for highly permeable and moderately permeable reference compounds.

FIG. 5 illustrates a linear regression analysis for the relationship between human absorption data and apparent permeability for Caco-2 monolayer cells.

### DETAILED DESCRIPTION

### Definitions

The following description provides context and examples, but should not be interpreted to limit the scope of the disclosure covered by the claims that follow in this specification or in any other application that claims priority to this specification. No single component or collection of components is essential or indispensable. For example, some embodiments one or more variables, such as Y or Y and Q may be omitted. Any feature, structure, component, material, step, or method that is described and/or illustrated in any embodiment in this specification can be used with or instead of any feature, structure, component, material, step, or method that is described and/or illustrated in any other embodiment in this specification.

The term “bioavailability,” as used herein is the proportion of a drug or other substance which enters the circulation when introduced into the body and so is able to have an active effect.

The terms “cancer” and “tumor” are used interchangeably herein and shall be given their ordinary meaning and shall also refer to cells which exhibit relatively autonomous growth, so that they exhibit an aberrant growth phenotype characterized by a significant loss of control of cell proliferation. In general, cells of interest for detection or treatment in the present application include precursors, precancerous (e.g., benign), malignant, pre-metastatic, metastatic, and non-metastatic cells.

The term “diagnosis,” as used herein shall be given its ordinary meaning and shall also include determination of a subject's susceptibility to a disease or disorder, determination as to whether a subject is presently affected by a disease or disorder, prognosis of a subject affected by a disease or disorder (e.g., identification of cancer or cancerous states, stages of cancer, or responsiveness of cancer to therapy), and use of therametrics (e.g., monitoring a subject's condition to provide information as to the effect or efficacy of therapy).

The term “lag time.” as used herein is a time period during which the percentage of the active pharmaceutical ingredient that is released from the pharmaceutical composition is equal to or less than about 0, 0.5, 1, 2, 3, 5, 8, 10, or ranges including and/or spanning the aforementioned values, where percentage is based on the total amount of the active pharmaceutical ingredient present in the pharmaceutical composition.

The term “monocation,” as used herein is a molecule that is bonded, or otherwise connected, to one proton that is not part of the molecule's essential structure, the molecule has a formal charge of +1. The term “dication,” as used herein, is a molecule that is bonded, or otherwise connected, to two protons that are not part of the molecule's essential structure, the molecule has a formal charge of +2. The term “polycation,” as used herein, is a molecule that is bonded, or otherwise connected, to two or more protons that are not part of the molecule's essential structure, the molecule has a formal charge that is positive and is equal to the number of protons bonded to the molecule.

The term “organic acid.” as used herein is a water-soluble, pharmaceutically acceptable carboxylic acid or sulfonic acid configured to increase the rate and/or the extent of dissolution of an active pharmaceutical ingredient in an aqueous solution.

The term “permeability,” as used herein is the rate at which a substance, such as a drug, passes through body tissue, such as intestinal membranes. Permeability can be classified either as apparent permeability or as effective permeability. The term “apparent permeability,” as used herein relates to permeability values obtained with in vitro experiments. The term “effective permeability,” as used herein relates to permeability values obtained ex-vivo methods such as single-pass intestinal perfusion.

The terms “plasma profile,” “plasma concentration,” “Cₘₐₓ,” and “Cₘᵢₙ” as used herein refer to the concentration of drug in the plasma of a subject, generally expressed as mass per unit volume, e.g., nanograms per milliliter (ng/mL), or moles per unit volume, micromoles per liter (μmole/L)

The terms “pharmaceutically acceptable salt” and “salt” as used herein refer to a salt of a compound that does not cause significant irritation to an organism to which it is administered and does not abrogate the biological activity and properties of the compound. In several embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

The term “release rate,” as used herein refers to the quantity of drug released in vitro or in vivo from a composition per unit time. The units of quantity are often expressed as, e.g., % of the total dose.

As used herein, “subject,” “host,” “patient,” and “individual” are used interchangeably and shall be given their ordinary meaning in the art and shall also refer to an organism that has cancer and/or leukemia. This includes mammals, e.g., a human, a non-human primate, ungulates, canines, felines, equines, mice, rats, and the like. The term “mammal” includes both human and non-human mammals.

The terms “therapeutically effective amount” and “effective amount” refer to the amount of active pharmaceutical ingredient necessary to provide the desired pharmacologic result. In practice, the therapeutically effective amount will vary widely depending on the severity of the disease condition, age of the subject, and the desired therapeutic effect.

The terms “treatment,” “treating,” “treat,” and the like shall be given their ordinary meaning and shall also include herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. The terms “treatment,” as used herein shall be given its ordinary meaning and shall also cover any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; and/or (c) relieving the disease symptom, e.g., causing regression of the disease or symptom.

As used herein, the term “weight percent,” when referring to a component, is the weight of the component divided by the weight of the composition that includes the component, multiplied by 100%. For example, the weight percent of component A when 5 grams of component A is added to 95 grams of component B is 5% (e.g., 5 g A/(5 g A+95 g B)×100%).

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the general description and the following detailed description are exemplary and explanatory only and are not restrictive. The term “and/or” denotes that the provided possibilities can be used together or be used in the alternative. Thus, the term “and/or” denotes that both options exist for that set of possibilities.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the disclosure. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Introduction

Histamine receptors are a promising molecular therapeutic target for many types of cancer treatment. Some histamine receptors are differentially expressed in tumors compared with normal tissues, and this expression is associated with clinicopathological characteristics in most cancer types, suggesting that histamine receptors might represent a novel cancer biomarker. However, the availability of pharmaceutical compositions suitable for oral administration of histamine is limited by the acidic conditions of the gastro-intestinal (GI) tract. Additionally, oral administration of a pharmaceutical composition containing histamine by existing methods can be very harmful to the human GI tract. Several embodiments disclosed herein pertain to compounds that achieve one or more of these advantages (or others). Several embodiments disclosed herein pertain to pharmaceutical compositions that address one or more deficiencies of known drug substances. Several embodiments disclosed herein pertain to a GI-safe, orally administered pharmaceutical composition containing histamine the treatment of solid tumors and leukemias.

### Delayed Release Beads

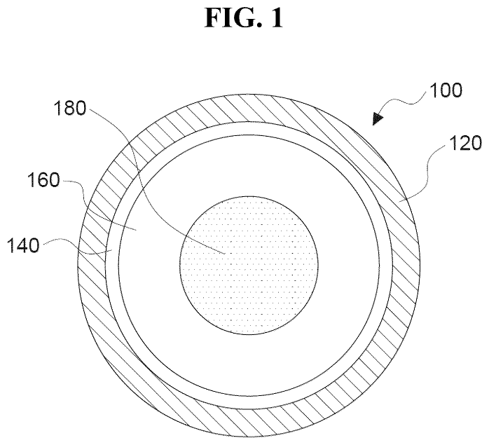

Several embodiments of the present disclosure relate to a pharmaceutical composition comprising delayed release beads. As shown in FIG. 1, in some embodiments, a delayed release bead 100 includes a delayed release coating 120, a sealant coating layer 140, an amorphous layer 160, and an inert particle core 180.

The amorphous layer 160 of the delayed release beads includes a solid dispersion of a therapeutically effective dosage of at least one active pharmaceutical ingredient (API). The terms “active pharmaceutical ingredient” and “API” are used interchangeably herein with each other and with the terms “drug” and “therapeutic agent.” The term “API” includes both a single API and a combination of several APIs as in cases where the solid dispersion comprises more than one API. As used herein, API includes a distinct compound, pharmaceutically acceptable salts, polymorphs, stereoisomers, solvates, esters, and any combination of the foregoing.

In several embodiments, the API includes histamine, a histamine salt, a histamine derivative, a salt of a histamine derivative, and any combination of the foregoing. In some embodiments, the API is histamine. In certain embodiments, the API is a histamine salt. In other embodiments, the API is a histamine derivative. In other embodiments, the API is a salt of a histamine derivative.

In several embodiments, the solid dispersion includes at least one API and at least one solubility-enhancing polymer. The term “solid dispersion” as used herein is a substantially amorphous active pharmaceutical ingredient dispersed in a polymeric matrix. In several embodiments, the API and the solubility-enhancing polymer are substantially molecularly dispersed in the solid state. The term “substantially amorphous” means that less than 40% of the API forms a separate crystalline phase in the polymeric matrix. In other embodiments, “substantially amorphous” means that less than 30%, less than 20%, less than 10%, less than 5%, or less than 1% of the API forms a separate crystalline phase in the polymeric matrix. Alternatively stated, at least 60%, at least 70%, at least 80%, at least 90%, at least 95%, or at least 99% of the API is in the amorphous state. The term “substantially molecularly dispersed” means that less than 40% of the API forms a separate crystalline phase in the polymeric matrix, and the remainder of the API is dissolved in the polymeric matrix. In other embodiments, “substantially molecularly dispersed” means that less than 30%, less than 20%, less than 10%, less than 5%, or less than 1% of the API forms a separate crystalline phase in the polymeric matrix. The solid dispersions of the present disclosure include combinations of “substantially molecularly dispersed” and “substantially amorphous” API in the polymeric matrix, provided that no more than 40% of the API, and in some embodiments or more than 30%, no more than 20%, or more than 10%, no more than 5%, or no more than 1% of the API forms a crystalline phase in the polymeric matrix. As used herein, the term “solubility-enhancing polymer” is a water-soluble polymer capable of forming a solid dispersion of the API. In a non-limiting example, the solid dispersion may be formed by dissolving the API and solubility-enhancing polymer together in the same solvent system and removing the solvent under appropriate conditions in a subsequent step. The API is maintained as a molecular dispersion or in amorphous form during storage, transportation, and distribution of the pharmaceutical composition containing the solid dispersion of the API and the solubility-enhancing polymer. The solid dispersion can be in the form of particles (e.g., granules, pellets, beads, and the like).

Suitable solubility-enhancing polymers useful in the pharmaceutical compositions of the present disclosure include but are not limited to polyvinylpyrrolidone (PVP or povidone), copolymers of vinyl acetate/vinylpyrrolidone (e.g. Kollidon VA 64), methylcellulose, hydroxypropyl cellulose, hydroxypropyl methylcellulose (hypromellose), hydroxypropylmethylcellulose acetate succinate (HPMCAS), polyethylene oxide, polyethylene glycol, and cyclodextrin. The type and amount of solubility-enhancing polymer is selected so that the combination of API and solubility-enhancing polymer form a solid dispersion as defined herein. Some of the solubility-enhancing polymers useful for preparing solid solutions/dispersions are conventionally used as binders. However, to provide a solid dispersion of the active pharmaceutical ingredient, the ratio of solubility-enhancing polymer to active pharmaceutical ingredient is generally significantly higher than the ratio of polymeric binder to active pharmaceutical ingredient in conventional pharmaceutical formulations. (In conventional pharmaceutical formulations, the ratio of polymeric binder to active pharmaceutical ingredient is typically less than 1/9, for example about 1/50 to about 1/20.) In one embodiment, the ratio of solubility-enhancing polymer to active pharmaceutical ingredient in the solid dispersion ranges from 9/1 to ⅙ (by weight). In another embodiment, the ratio the solubility-enhancing polymer to active pharmaceutical ingredient in the solid dispersion ranges from about 3/1 to about ⅓ (by weight). In yet another embodiment, the ratio the solubility-enhancing polymer to active pharmaceutical ingredient in the solid dispersion ranges from about 2/1 to about ½ (by weight), or about 1/1.

In several embodiments, the solid dispersion includes at least one pharmaceutically acceptable solid buffer organic acid. The pharmaceutically acceptable solid buffer organic acid can further improve or modulate the release profile of the API (e.g. rate and extent of release). Suitable pharmaceutically acceptable organic acids useful in the compositions of the present disclosure include, but are not limited to, citric acid, fumaric acid, aspartic acid, tartaric acid and succinic acid. In some embodiments, the solid dispersion of API and solubility enhancing polymer includes at least one pharmaceutically acceptable organic acid in an amount ranging from about 10-90% of the weight of the solid dispersion. In other embodiments, the amount organic acid ranges from 25-75% by weight of the solid dispersion.

In several embodiments, the amorphous layer 160 is present in the delayed release bead 100 at a weight percent of equal to or less than about: 10%, 20%, 25%, 30%, 32%, 34%, 36%, 38%, 40%, 45%, 50%, 60%, or ranges including and/or spanning the aforementioned values.

The inert particle core 180 may be any pharmaceutically acceptable inert material, non-limiting examples of which include sugar spheres or beads (e.g., Celpheren), cellulose spheres, and silicon dioxide spheres. The insert particle core 180 has a suitable particle size distribution from about 20-25 mesh to 35-40 mesh for coated beads used in a capsule formulation. In several embodiments, the solid dispersion can be layered on to the inert particle core 180. For example, the API and solubility-enhancing polymer can be dissolved in a pharmaceutically acceptable solvent (or mixture of solvents) and coated onto the inert particle core 180. Upon removal of the solvent, the solid dispersion is formed as a coating on the inert particle core 180. The thickness of the solid dispersion layer and relative amounts of API and solubility-enhancing polymer can be adjusted to provide a therapeutically effective amount of the API. For example, the inert particle core 180 layered with a solid dispersion of the active pharmaceutical ingredient can contain 2% to about 50% by weight of the API, based on a total weight of the inert particle core 180 and the API.

In several embodiments, the inert particle core 180 is present in the delayed release bead 100 at a weight percent of equal to or less than about: 20%, 30%, 40%, 50%, 53%, 56%, 60%, 62%, 64%, 66%, 68%, 70%, 75%, 80%, 85%, or ranges including and/or spanning the aforementioned values.

The delayed release coating 120 includes a water insoluble polymer and an enteric polymer. Suitable water insoluble polymers include cellulose derivatives (e.g. ethylcellulose), polyvinyl acetate (Kollicoat SR30D from BASF), neutral copolymers based on ethyl acrylate and methylmethacrylate, copolymers of acrylic and methacrylic acid esters with quaternary ammonium groups, such as Eudragit NE, RS or RS30D, RL or RL30D and the like. The term “enteric polymer” as used herein, is a material that is insoluble at pH levels found in the stomach, and is soluble at pH levels found in the intestinal tract. Suitable enteric polymers include acid substituted cellulose esters (e.g., cellulose acetate phthalate, hydroxypropyl methylcellulose phthalate, hydroxypropyl methylcellulose acetate succinate), polyvinyl acetate phthalate, pH-sensitive methacrylic acid-methamethacrylate copolymers and shellac. Commercially available enteric polymers suitable for use herein are sold under the trade name “Eudragit” (e.g., Eudragit LI 00, S100, L30D) manufactured by Rhom Pharma, Cellacefate (cellulose acetate phthalate) from Eastman Chemical Co., Aquateric (cellulose acetate phthalate aqueous dispersion) from FMC Corp. and Aqoat (hydroxypropyl methylcellulose acetate succinate aqueous dispersion) from Shin Etsu K. K. In several embodiments, the delayed release coating 120 can be a coating described in U.S. Pat. No. 6,627,223, herein incorporated by reference for all purposes. In several embodiments, the delayed release coating 120 can be plasticized. Representative examples of suitable plasticizers that can be used to plasticize the delayed release coating 120 include triacetin, tributyl citrate, triethyl citrate, acetyl tri-n-butyl citrate, diethyl phthalate, castor oil, dibutyl sebacate, acetylated monoglycerides and the like or mixtures thereof. The plasticizer, when present, can comprise about 3 to 30% of the total weight of the delayed release coating 120. In one embodiment, the plasticizer comprises about 10 to 25% of the total weight of the delayed release coating 120. The type and amount of plasticizer depends on the nature of the water insoluble and enteric polymers of the delayed release coating 120, and the nature of the coating system (e.g., aqueous or solvent based, solution or dispersion based, and the total solids content of the coating system).

In several embodiments, the delayed release coating 120 is insoluble at pH equal to or less than about 3.5. The solubility of the delayed release coating 120 is reported in units of micrograms per milliliter (μg/mL). In several embodiments, the delayed release coating 120 has a solubility in μg/mL equal to or less than about: 0.001, 0.005, 0.01, or ranges including and/or spanning the aforementioned values, at pH equal to or less than about 3.5, 3.8, 4.0, or ranges including and/or spanning the aforementioned values. In some embodiments, the delayed release coating 120 is soluble at pH equal to or greater than about 6. In some embodiments, the delayed release coating 120 is soluble at pH from about 6 to about 8.5. In several embodiments, the delayed release coating 120 has a solubility equal to or greater than about 100 μg/mL at pH equal to or greater than about 6. In several embodiments, the delayed release coating 120 has a solubility from about 30 μg/mL to about 100 μg/mL at pH from about 6 to about 8.5. In several embodiments, the delayed release coating 120 has a solubility in μg/mL equal to or greater than about: 10, 20, 30, 40, 50, 60, 70, 80, 90, 100, 110, 120, 150, 200, or ranges including and/or spanning the aforementioned values, at pH equal to or greater than about 5.7, 5.8, 5.9, 6, 6.1, 6.2, 6.3, 6.5, 6.8, 7 7.5, 8, 8.5, 9, 10 or ranges including and/or spanning the aforementioned values.

The delayed release coating 120 modulates the release of the API from the pharmaceutical composition. The combination of the delayed release coating 120 and the solid dispersion of the API provides an improved release profile of the API compared to the release profile obtained by a composition in which the API is not part of a solid dispersion and/or lacks a delayed release coating 120. In some embodiments, a rate of release of the API from the pharmaceutical composition is determined by the solubility of the delayed release coating 120, the amount of the delayed release coating 120 present in the delayed release bead 100, the weight ratio of water insoluble polymer to enteric polymer in the delayed release coating 120, and any combination of the foregoing. In several embodiments, the rate of release of the API from the pharmaceutical composition can be approximately constant over about 0.5, 1, 2, 3, 5, 7, 9, 10 hours (h), or ranges including and/or spanning the aforementioned values. In several embodiments, the maximum rate of release of the API from the pharmaceutical composition occurs about 0.1, 0.25, 0.5, 0.75, 1, 1.25, 1.5, 1.75, 2, 2.25, 2.5, 3, 3.5, 4, 4.5, 5, 6, 7, 8, 9, 10 h after the composition is administered, or ranges including and/or spanning the aforementioned values.

In some embodiments, the solid dispersion, the delayed release coating 120, the pharmaceutical composition, or any combination of the above comprises a lag time as described elsewhere herein. In several embodiments, the lag time is about 0.1, 0.25, 0.5, 0.75, 1, 1.25, 1.5, 1.75, 2, 2.25, 2.5, 3, 3.5, 4, 4.5, 5, 5.5, 6, 7, 8, 9, 10 h, or ranges including and/or spanning the aforementioned values.

In several embodiments, the delayed release coating 120 is present in the delayed release bead 100 at a weight percent of equal to or less than about: 1%, 5%, 8%, 10%, 12%, 15%, 20%, 25%, 28%, 30%, 35%, 40%, 50% or ranges including and/or spanning the aforementioned values. In several embodiments, a weight ratio of water insoluble polymer to enteric polymer in the delayed release coating 120 is equal to about: 1/9, ⅛, 1/7, ⅙, ⅕, ¼, ⅓, ½, 2/1, 3/1, 4/1, 5/1, 6/1, 7/1, 8/1, 9/1, or ranges including and/or spanning the aforementioned values.

In several embodiments, the sealant coating layer 140 comprises a protective coating, a sealant coating, a compressible coating, an enteric coating, a taste-masking coating, and any combination of the foregoing. In several embodiments, the sealant coating layer 140 includes at least one protective coating or sealant coating selected from hydroxypropylmethylcellulose, hydroxypropylcellulose, ethylcellulose, and any combination of the foregoing. In several embodiments, the sealant coating layer 140 comprises OPADRY Clear, PHARMACOAT 603, or both. The sealant coating layer 140 can be applied between the inert particle core 180 and the amorphous layer 160, between the amorphous layer 160 and the delayed release coating 120, on top of the delayed release coating 120, and any combination of the foregoing. In several embodiments, the sealant coating layer 140 can be applied in multiple layers. In other embodiments, the sealant coating layer 140 comprises an enteric polymer as described elsewhere herein.

In several embodiments, the sealant coating layer 140 is present in the delayed release bead 100 at a weight percent of equal to or less than about: 0.1%, 0.5%, 0.8%, 1%, 1.2%, 1.5%, 2%, 2.5%, 2.8%, 3%, 5%, 10%, 20%, or ranges including and/or spanning the aforementioned values.

### Enteric Tablet

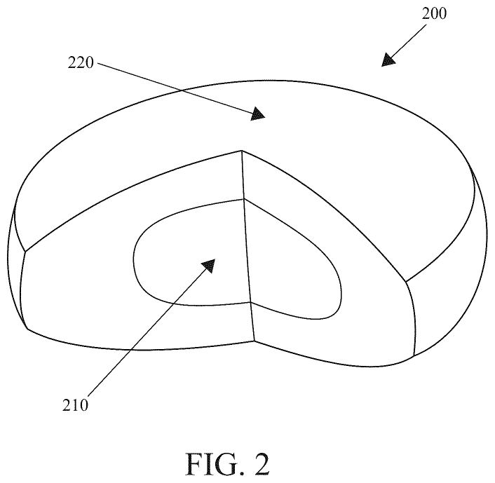

Several embodiments of the present disclosure relate to a pharmaceutical composition that includes an enteric tablet 200. As shown in FIG. 2, in some embodiments, the enteric tablet 200 includes a compressed tablet 210 and a polymeric coating 220.

The compressed tablet 210 has a characteristic shape and an outer surface. In several embodiments, the shape of the compressed tablet 220 can be round, oval, flat, oblong, and any combination of the foregoing. In several embodiments, the compressed tablet 220 has a largest dimension (length, width, depth, diameter, etc.) of about 0.5, 1, 2, 2.3, 2.6, 2.9, 3.2, 3.5, 3.8, 4.1, 4.3, 4.7, 5, 5.5, 6, 6.5, 7, 7.5, 8, 8.5, 9, 9.5 10, 12, 14, 16, 18, 20 millimeters (mm) or ranges including and/or spanning the aforementioned values. The compressed tablet 220 includes a therapeutically effective dosage of at least one active pharmaceutical ingredient (API), as described elsewhere herein. In several embodiments, the API includes histamine, a histamine salt, a histamine derivative, a salt of a histamine derivative, and any combination of the foregoing. In some embodiments, the API is histamine. In certain embodiments, the API is a histamine salt. In other embodiments, the API is a histamine derivative. In other embodiments, the API is a salt of a histamine derivative. In several embodiments, the API is granulated. In several embodiments, the API is granulated histamine. In several embodiments, the API is a granulated histamine salt. In other embodiments, the API is a granulated histamine derivative. In other embodiments, the API is a granulated salt of a histamine derivative. In several embodiments, the compressed tablet 220 has a total mass of about 1, 5, 10, 15, 20, 30, 50, 80, 100, 150, 200, 250, 300, 500, 700, 1000, 1500, 2000 milligrams (mg) or ranges including and/or spanning the aforementioned values. In several embodiments, the compressed tablet 220 has a total mass of the API of about 1, 5, 10, 15, 20, 30, 50, 80, 100, 150, 200, 250, 300, 500, 700, 1000, 1500, 2000 milligrams (mg) or ranges including and/or spanning the aforementioned values. In several embodiments, the compressed tablet 210 is completely encapsulated, or otherwise surrounded or sealed, by the polymeric coating 220 such that the compressed tablet 210 is not exposed to the surroundings of the polymeric coating 220.

The polymeric coating 220 is located and adhered to on the outer surface of the compressed tablet 210. In several embodiments, the polymeric coating 220 comprises an enteric coating. The term “enteric coating,” as used herein, is a coating that does not dissolve in the stomach and dissolves readily in the small intestine. The polymeric coating 220 can be an enteric coating that: prevents the compressed tablet 210 from being dissolved in the acid environment of the stomach; delays the compressed tablet 210 from being dissolved until it is in the small intestine; and/or allows the compressed tablet 210 to be dissolved in the small intestine.

In several embodiments, the polymeric coating 220 includes at least one fat, at least one fatty acid, at least one plant fiber, at least one plastic, at least one shellac, at least one shellac derivative, at least one wax, and any combination of the foregoing. In several embodiments, the polymeric coating 220 includes a water-soluble polymer and/or a water-insoluble polymer. In several embodiments, the polymeric coating 220 includes cellulose acetate phthalate, carboxymethylcellulose, cellulose acetate trimellitate, ethylcellulose, hydroxyl propyl methyl cellulose phthalate, hydroxyl propyl methyl cellulose acetate succinate, hydroxyethylcellulose polyacrylic acid, a methacrylic acid copolymer (e.g., polymethacrylate), methylcellulose, polyvinyl acetate phthalate, and any combination of the foregoing. In several embodiments, the polymeric coating 220 includes a blend of a wax and polymethacrylate. In several embodiments, the polymeric coating 220 includes one or more ingredients selected from surfactants, plasticizers, antifoaming agents, solubilizing agents and coloring agents.

In several embodiments, the polymeric coating 220 insoluble at pH equal to or less than about 3.5. The solubility of the polymeric coating 220 is reported in units of micrograms per milliliter (μg/mL). In several embodiments, the polymeric coating 220 has a solubility in μg/mL equal to or less than about: 0.001, 0.005, 0.01, or ranges including and/or spanning the aforementioned values, at pH equal to or less than about 3.5, 3.8, 4.0, or ranges including and/or spanning the aforementioned values. In some embodiments, the polymeric coating 220 is soluble at pH equal to or greater than about 6. In some embodiments, the polymeric coating 220 is soluble at pH from about 6 to about 8.5. In several embodiments, the polymeric coating 220 has a solubility equal to or greater than about 100 μg/mL at pH equal to or greater than about 6. In several embodiments, the polymeric coating 220 has a solubility from about 30 μg/mL to about 100 μg/mL at pH from about 6 to about 8.5. In several embodiments, the polymeric coating 220 has a solubility in μg/mL equal to or greater than about: 10, 20, 30, 40, 50, 60, 70, 80, 90, 100, 110, 120, 150, 200, or ranges including and/or spanning the aforementioned values, at pH equal to or greater than about 5.7, 5.8, 5.9, 6, 6.1, 6.2, 6.3, 6.5, 6.8, 7 7.5, 8, 8.5, 9, 10 or ranges including and/or spanning the aforementioned values. In several embodiments, the polymeric coating 220 is insoluble at pH equal to or less than about 3.5 and is soluble at pH equal to or greater than about 6.

In several embodiments, a thickness of the polymeric coating 220 is about 0.001, 0.005, 0.01, 0.015, 0.02, 0.03, 0.04, 0.05, 0.06, 0.07, 0.08, 0.09, 0.1, 0.2, 0.3, 0.4, 0.5, 0.7, 0.9, 1, 1.3, 1.6, 1.9, 2, mm or ranges including and/or spanning the aforementioned values.

In several embodiments, the pharmaceutical composition provides the therapeutically effective plasma concentration of the API measured in micromoles per liter in μmole/L equal to or greater than about 0.01, 0.05, 0.1, 0.15, 0.17, 0.18, 0.19, 0.2, 0.21, 0.22, 0.23, 0.24, 0.25, 0.3, 0.5, 1, 2, 5, 10, 20, 50, 100, or ranges including and/or spanning the aforementioned values. In several embodiments, the pharmaceutical composition provides the therapeutically effective plasma concentration of the API equal to or greater than about 0.2 μmole/L. In several embodiments, the pharmaceutical composition provides the therapeutically effective plasma concentration of the API for equal to or greater than about 0.1, 0.25, 0.5, 0.75, 1, 1.25, 1.5, 1.75, 2, 2.25, 2.5, 3, 3.5, 4, 4.5, 5, 6, 7, 8, 9, 10 h, or ranges including and/or spanning the aforementioned values. In several embodiments, the pharmaceutical composition provides the therapeutically effective plasma concentration of the API for about 0.25 to about 5 h, or about 0.5 to about 3 h, or about 0.5 to about 1.25 h. The therapeutically effective plasma concentration of the API is determined in accordance with United States Pharmacopoeia (USP) dissolution methodology using a two-stage dissolution medium (first 2 hours in 0.1N HCl followed by testing in a buffer at pH 6.8). In some embodiments, a modeling exercise is typically performed using the pharmacokinetic parameters for the drug using the software program, WINNONLIN Standard Version 2.1 or equivalent (e.g., GASTROPLUS to fit a 1-compartment first order model with a lag time assuming first order elimination kinetics. The primary parameters are then input into another program, Stella Version 6.01 using a previously established model with slight modifications. Different in vitro release profiles are generated, and from target once-daily release profiles, desired in vitro release (medium, target and fast) profiles are generated by deconvolution.

In several embodiments, the histamine salt comprises a histamine monocation, a histamine polycation, and any combination of the foregoing. In several embodiments, the histamine salt comprises a histamine dication. In several embodiments, the histamine salt comprises at least one anion selected from acetate, aspartate, citrate, formate, fumarate, halide, malate, nitrate, nitrite, phosphite, phosphate, succinate, sulfate, sulfite, tartrate, and any combination of the foregoing. In several embodiments, the histamine salt comprises histamine dihydrohalide or histamine phosphate. In several embodiments, the histamine salt comprises histamine dihydrochloride.

The term “histamine derivative,” as used herein, is a structural analog of histamine and/or a synthetically derived analog of histamine. In several embodiments, the histamine derivative comprises C₁₋₆ alkyl histamine. In some embodiments, the histamine derivative comprises N—(C₁₋₆ alkyl)histamine, 1-(C₁₋₆ alkyl)histamine, 2-(C₁₋₆ alkyl)histamine, 4-(C₁₋₆ alkyl)histamine, 5-(C₁₋₆ alkyl)histamine, alpha-(C₁₋₆ alkyl)histamine, and any combination of the foregoing. In several embodiments, the histamine derivative comprises N-methylhistamine, 1-methylhistamine, 2-methylhistamine, 4-methylhistamine, 5-methylhistamine or alpha-methylhistamine. In several embodiments, the histamine derivative comprises N-methylhistamine. In several embodiments, the histamine derivative comprises alpha-methylhistamine.

In several embodiments, the salt of the histamine derivative comprises C₁₋₆ alkyl histamine monocation, C₁₋₆ alkyl histamine polycation and any combination of the foregoing. In several embodiments, the salt of the histamine derivative comprises C₁₋₆ alkyl histamine dication. In several embodiments, the salt of the histamine derivative comprises N—(C₁₋₆ alkyl)histamine dication, 1-(C₁₋₆ alkyl)histamine dication, 2-(C₁₋₆ alkyl)histamine dication, 4-(C₁₋₆ alkyl)histamine dication, 5-(C₁₋₆ alkyl)histamine dication, alpha-(C₁₋₆ alkyl)histamine dication, and any combination of the foregoing. In several embodiments, the salt of the histamine derivative comprises at least one anion selected from acetate, aspartate, citrate, formate, fumarate, halide, malate, nitrate, nitrite, phosphite, phosphate, succinate, sulfate, sulfite, tartrate, and any combination of the foregoing. In several embodiments, the salt of the histamine derivative comprises N-methylhistamine dihydrochloride, 1-methylhistamine dihydrochloride, 2-methylhistamine dihydrochloride, 4-methylhistamine dihydrochloride, 5-methylhistamine dihydrochloride, alpha-methylhistamine dihydrochloride, and any combination of the foregoing. In several embodiments, the salt of the histamine derivative comprises N-methylhistamine dihydrochloride. In several embodiments, the salt of the histamine derivative comprises alpha-methylhistamine dihydrochloride.

In several embodiments, the solid dispersion comprises at least one a pharmaceutically acceptable solid buffer, the buffer including a pharmaceutically acceptable organic acid or pharmaceutically acceptable alkaline buffer. In several embodiments, the organic acid comprises aspartic acid, formic acid, fumaric acid, acetic acid, succinic acid, lactic acid, malic acid, tartaric acid, citric acid, ascorbic acid, nicotinic acid, methanesulfonic acid, ethanesulfonic acid, p-toluensulfonic acid, salicylic acid, naphthalenesulfonic acid and any combination of the foregoing. In several embodiments, the organic acid comprises aspartic acid. In several embodiments, the organic acid comprises fumaric acid. In several embodiments, the organic acid comprises tartaric acid. In several embodiments, the alkaline buffer comprises a cation comprising an alkali metal, an alkaline earth metal and any combination of the foregoing and an anion comprising acetate, citrate, formate, halide, malate, nitrate, nitrite, phosphite, phosphate, succinate, sulfate, sulfite, tartrate, and any combination of the foregoing.

In some embodiments, the API includes at least one histamine receptor agonist. In several embodiments, the histamine receptor agonist is selected from the group consisting of a histamine H1 receptor agonist, a histamine H2 receptor agonist, a histamine H3 receptor agonist, a histamine H4 receptor agonist, and any combination of the foregoing. In several embodiments, the API is a histamine H2 receptor agonist. In certain embodiments, the API does not include an antihistamine. In other embodiments, the API does not include a histamine receptor antagonist. In several embodiments, the API does not include an H1 receptor antagonist, an H2 receptor antagonist, an H3 receptor antagonist, an H4 receptor antagonist, and any combination of the foregoing. In several embodiments, the API includes at least one histamine structural analog having H2 receptor agonist activity.

In several embodiments, the API inhibits the production or release of intracellular hydrogen peroxide. In several embodiments, the API inhibits the production or release of intracellular hydrogen peroxide by monocytes. In several embodiments, the plasma concentration of the API is sufficient to inhibit the production or release of intracellular hydrogen peroxide by monocytes. In an embodiment, the inhibition occurs when the API binds histamine H2 receptors. In several embodiments, the inhibition occurs when the API binds histamine H2 receptors selectively. In several embodiments, the API has an affinity for histamine H2 receptors that is higher compared to the affinity for any other histamine receptor. In several embodiments, the API has affinity for histamine H2 receptors that is higher compared to the affinity for a H1 receptor, a H3 receptor, a H4 receptor, and any combination of the foregoing.

In several embodiments, the API has a solubility equal to or greater than about: 40%, 50%, 60%, 70%, 75%, 80%, 85%, 87%, 89%, 90%, 91%, 92%, 93%, 94%, 95%. 96%, 97%, 98%, 99%, 99.5%, or ranges including and/or spanning the aforementioned values. In several embodiments, the API has a solubility from about 70% to 99.5%, or about 85% to 99%, or about 95% to 99%. In several embodiments, the pharmaceutical composition has a solubility equal to or greater than about: 40%, 50%, 60%, 70%, 75%, 80%, 85%, 87%, 89%, 90%, 91%, 92%, 93%, 94%, 95%, 96%, 97%, 98%, 99%, 99.5%, or ranges including and/or spanning the aforementioned values. In several embodiments, the solubility of histamine dihydrochloride is equal to or greater than about: 40%, 50%, 60%, 70%, 75%, 80%, 85%, 87%, 89%, 90%, 91%, 92%, 93%, 94%, 95%, 96%, 97%, 98%, 99%, 99.5%, or ranges including and/or spanning the aforementioned values. In several embodiments, the solubility of the API, the pharmaceutical composition, and/or histamine dihydrochloride is measured at a pH of about 5, 6, 6.6, 6.8, 7, 7.2, 7.4, 7.6, 8, 8.2, 8.4 or ranges including and/or spanning the aforementioned values. In several embodiments, the solubility of the API, the pharmaceutical composition, and/or histamine dihydrochloride is measured at a pH from about 7 to 7.8 or about 7.2 to 7.6.

In several embodiments, the API has an apparent permeability expressed in microcentimeters per second (μcm/s) equal to or greater than about 1, 3, 5, 13, 15, 16, 17, 18, 19, 20, 21, 22, 23, 24, 28, 30, 40, or ranges including and/or spanning the aforementioned values. In several embodiments, the API has an apparent permeability from about 1 to 40 μcm/s, about 12 to 24 μcm/s, about 16 to 20 μcm/s. In several embodiments, the pharmaceutical composition has an apparent permeability expressed in μcm/s equal to or greater than about 1, 3, 5, 13, 15, 16, 17, 18, 19, 20, 21, 22, 23, 24, 28, 30, 40, or ranges including and/or spanning the aforementioned values. In several embodiments, the pharmaceutical composition has an apparent permeability from about 1 to 40 μcm/s, about 12 to 24 μcm/s, about 16 to 20 μcm/s. In several embodiments, histamine dihydrochloride has an apparent permeability expressed in μcm/s equal to or greater than about 1, 3, 5, 13, 15, 16, 17, 18, 19, 20, 21, 22, 23, 24, 28, 30, 40, or ranges including and/or spanning the aforementioned values. In several embodiments, histamine dihydrochloride has an apparent permeability from about 1 to 40 μcm/s, about 12 to 24 μcm/s, about 16 to 20 μcm/s. In several embodiments, the apparent permeability of the API, the pharmaceutical composition, and/or histamine dihydrochloride is measured across the small intestine. In several embodiments, the apparent permeability of the API, the pharmaceutical composition, and/or histamine dihydrochloride is measured at a pH of about 5, 6, 6.6, 6.8, 7, 7.2, 7.4, 7.6, 8, 8.2, 8.4 or ranges including and/or spanning the aforementioned values. In several embodiments, the apparent permeability of the API, the pharmaceutical composition, and/or histamine dihydrochloride is measured at a pH from about 7 to 7.8 or about 7.2 to 7.6.

In several embodiments, the API is absorbed in the small intestine in an amount equal to or greater than about: 40%, 50%, 60%, 70%, 75%, 80%, 81%, 82%, 83%, 84%, 85%, 86%, 87%, 88%, 89%, 90%, 95%, 98%, 99%, 99.5% of the therapeutically effective dosage, or ranges including and/or spanning the aforementioned values. In several embodiments, the API is absorbed in the small intestine in an amount from about 40% to 99.5%, or about 75% to 99%, or about 80% to 90%. In several embodiments, the pharmaceutical composition is absorbed in the small intestine in an amount equal to or greater than about: 40%, 50%, 60%, 70%, 75%, 80%, 81%, 82%, 83%, 84%, 85%, 86%, 87%, 88%, 89%, 90%, 95%, 98%, 99%, 99.5% of the therapeutically effective dosage, or ranges including and/or spanning the aforementioned values. In several embodiments the pharmaceutical composition is absorbed in the small intestine in an amount from about 40% to 99.5%, or about 75% to 99%, or about 80% to 90%. In several embodiments, histamine dihydrochloride is absorbed in the small intestine in an amount equal to or greater than about: 40%, 50%, 60%, 70%, 75%, 80%, 81%, 82%, 83%, 84%, 85%, 86%, 87%, 88%, 89%, 90%, 95%, 98%, 99%, 99.5% of the therapeutically effective dosage, or ranges including and/or spanning the aforementioned values. In several embodiments, histamine dihydrochloride is absorbed in the small intestine in an amount from about 40% to 99.5%, or about 75% to 99%, or about 80% to 90%.

In several embodiments, the bioavailability of the API is equal to or greater than about: 40%, 50%, 60%, 70%, 75%, 80%, 81%, 82%, 83%, 84%, 85%, 86%, 87%, 88%, 89%, 90%, 95%, 98%, 99%, 99.5% of the therapeutically effective dosage, or ranges including and/or spanning the aforementioned values. In several embodiments, the bioavailability of the API is from about 40% to 99.5%, or about 75% to 99%, or about 80% to 90%. In several embodiments, the bioavailability of the pharmaceutical composition is equal to or greater than about: 40%, 50%, 60%, 70%, 75%, 80%, 81%, 82%, 83%, 84%, 85%, 86%, 87%, 88%, 89%. 90%, 95%, 98%, 99%, 99.5% of the therapeutically effective dosage, or ranges including and/or spanning the aforementioned values. In several embodiments, the bioavailability of the pharmaceutical composition is from about 40% to 99.5%, or about 75% to 99%, or about 80% to 90%. In several embodiments, the oral bioavailability of the API is equal to or greater than about: 40%, 50%, 60%, 70%, 75%, 80%, 81%, 82%, 83%, 84%, 85%, 86%, 87%, 88%, 89%, 90%, 95%, 98%, 99%, 99.5% of the therapeutically effective dosage, or ranges including and/or spanning the aforementioned values. In several embodiments, the oral bioavailability of the API is from about 40% to 99.5%, or about 75% to 99%, or about 80% to 90%. In several embodiments, the oral bioavailability of the pharmaceutical composition is equal to or greater than about: 40%, 50%, 60%, 70%, 75%, 80%, 81%, 82%, 83%, 84%, 85%, 86%, 87%, 88%, 89%, 90%, 95%, 98%, 99%, 99.5% of the therapeutically effective dosage, or ranges including and/or spanning the aforementioned values. In several embodiments, the oral bioavailability of the pharmaceutical composition is from about 40% to 99.5%, or about 75% to 99%, or about 80% to 90%.

The pharmaceutical composition includes a therapeutically effective dosage of at least one active pharmaceutical ingredient (API). In several embodiments, the therapeutically effective dosage of the API in units of milligrams (mg) is equal to or less than about: 0.01, 0.03, 0.07, 0.1, 0.2, 0.3, 0.4, 0.5, 0.6, 0.7, 0.8, 0.9, 1, 1.1, 1.3, 1.6, 2, 3, 5, 8, 10, 15, 18, 20, 30, 50, 100, or ranges including and/or spanning the aforementioned values. In several embodiments, the therapeutically effective dosage of the API is from about 0.1 mg to about 10 mg, or from about 0.5 mg to about 8 mg.

The pharmaceutical compositions of the present disclosure can further comprise additional pharmaceutically acceptable ingredients or excipients. Examples of suitable excipients for use in the compositions or dosage forms include fillers, diluents, glidants, disintegrants, binders, lubricants etc. Other pharmaceutically acceptable excipients include acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents etc.

Examples of suitable fillers, diluents and/or binders include lactose, microcrystalline cellulose, hydroxypropylcellulose, hydroxypropyl methylcellulose, methyl cellulose polymers hydroxyethylcellulose, sodium carboxymethylcellulose, carboxymethylene, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, collagen etc.

Specific examples of diluents include e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Specific examples of disintegrants include e.g. alginic acid or alginates, microcrystalline cellulose, low-substituted hydroxypropyl cellulose and other cellulose derivatives, croscarmellose sodium, crospovidone, polacrillin potassium, sodium starch glycolate, starch, pregelatinized starch, carboxymethyl starch. Specific examples of binders include e.g., acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, polyethylene oxides, povidone, pregelatinized starch etc.

Specific examples of glidants and lubricants include stearic acid, magnesium stearate, calcium stearate or other metallic stearates, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients include e.g. flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents, agents for modified release etc.

Antioxidants used to improve long term chemical stability of the amorphous solid dispersion include ascorbic acid, ascorbyl palmitate, butylated hydroxyanisole, butylated hydroxytoluene, hypophosphorous acid, monothioglycerol, potassium metabisulfite, propyl gallate, sodium formaldehylde sulfoxylate, sodium metabisulfite, sodium thiosulfate, sulfur dioxide, tocopherol, tocopherol acetate, tocopherol hemisuccinate, TPGS or other tocopherol derivatives, etc.

The pharmaceutical compositions of the present disclosure can be formulated into various oral dosage forms, for example capsules (e.g., gelatin or HPMC capsules) and tablets. In one embodiment, the dosage forms may include one or more different types of delayed release beads (e.g., delayed release beads with different delayed release layers, or with different combinations of sealant and/or enteric layers). For example, delayed release beads having different delayed release layers can exhibit different lag time characteristics and/or different release rate characteristics, thereby providing the dosage form with different overall drug release characteristics.

In one embodiment, the dosage forms release the API over a period equal to or greater than about 0.1, 0.25, 0.5, 0.75, 1, 1.25, 1.5, 1.75, 2, 2.25, 2.5, 3, 3.5, 4, 4.5, 5, 6, 7, 8, 9, 10 h, or ranges including and/or spanning the aforementioned values. In several embodiments, the dosage forms release the API for about 0.25 to about 5 h, or about 0.5 to about 3 h, or about 0.5 to about 1.25 h. The drug release profile for the pharmaceutical compositions described herein can be evaluated in vitro using various dissolution testing methods, such as United States Pharmacopoeia Apparatus 1 (baskets@ 100 rpm) or Apparatus 2 (paddles @ 50 rpm) and a two-stage dissolution methodology, testing initially in 700 mL of 0.1N HCl for and then in 900 mL at pH 6.8. Drug/acid-release with time is determined by HPLC on samples obtained at selected intervals.

### Preparation of Pharmaceutical Compositions

Several embodiments of the present disclosure relate to a method of preparing a pharmaceutical composition comprising delayed release beads 100. Some embodiments relate to a method of preparing the delayed release beads 100. The solid dispersion of the API in the solubility-enhancing polymer can be prepared by dissolving the API and the solubility-enhancing polymer in a pharmaceutically acceptable solvent, or mixture of solvents, to form a solution. The solution of API and solubility-enhancing polymer is then dried under conditions which promote formation of a solid dispersion of the API in the solubility-enhancing polymer. In other words, the method includes removing the pharmaceutically acceptable solvent from the solution to form particles of the solid dispersion. In some embodiments, the particles comprise molecularly dispersed API and solubility-enhancing polymer. As discussed above, the formation of a molecularly dispersed solid dispersion is favored by relatively high levels of solubility-enhancing polymer relative to the API. In addition, solid dispersions can also be formed by rapidly removing the solvent from the solution of API and solubility enhancing polymer, for example by spray drying, or by coating the solution of API and solubility-enhancing polymer onto the inert particle core 180 to form a drug-layered bead, e.g. using fluidized bed coating methods. Alternatively, solid dispersions can also be prepared by dissolving the API into a melt of the solubility-enhancing polymer, e.g. by polymer extrusion methods, such as by compounding in a twin screw extruder. If necessary to obtain a suitable particle size (e.g., a particle size of less than 400 μm), particles of the solid dispersion can optionally be milled (to reduce the particle size), or granulated (e.g. rotogranulation, or granulation followed by extrusion-spheronization) in the presence of suitable excipients. The solid dispersion can also be formed into 1-2 mm diameter “mini-tablets”, e.g. formed by compressing particles of the solid dispersion, optionally with excipients such as compression aids, lubricants etc., using round beveled punches of the appropriate dimensions. In one embodiment, the solid dispersion is prepared by granulating the solubility enhancing polymer, the API and optionally other pharmaceutically acceptable excipients (e.g., binders, diluents, fillers) in a high-shear granulator, or a fluid bed granulator, such as Glatt GPCG granulator, and granulated to form agglomerates. The wet mass from the high-shear granulator can also be extruded and spheronized to produce spherical particles (pellets). When the solid dispersion is prepared by solvent processing methods, as discussed above, the pharmaceutically acceptable solvent can be a single solvent, or a mixture of solvents. Non-limiting examples of suitable solvents include water, ketones such as acetone, alcohols such as ethanol, and mixtures thereof (e.g., aqueous acetone, 95% ethanol, etc.).

Once prepared, the solid dispersion particles (e.g., spray-dried solid dispersion of API/polymer, drug-layered beads, granulated solid dispersion, mini-tablets, etc.) may be optionally coated with a protective sealant coat (e.g., PHARMACOAT 603 or OPADRY Clear).

The method includes dissolving the water insoluble polymer and the enteric polymer in a pharmaceutically acceptable coating solvent to form a delayed release coating solution, as described elsewhere herein. The method includes coating the particles of solid dispersion with the delayed release coating solution. Any suitable coating process can be used to apply the delayed release coating, for example fluidized bed coating methods, etc. In some embodiments, it is desirable to apply a plurality of coatings to the particles of solid dispersion, in addition to the delayed release coating. For example, in some embodiments, the particles are first coated with an enteric coating (e.g. comprising at least one enteric polymer, described herein, dissolved in a pharmaceutically acceptable solvent), dried to remove the coating solvents, then coated with the delayed release coating as described above. In other embodiments, the particles are coated with an enteric polymer coating, a delayed release coating, and then a second enteric polymer coating. In yet other embodiments, the particles are coated with a first delayed release coating, an enteric polymer coating, and then a second delayed release coating, wherein the first and second delayed release coatings are independently either the same or different. In still other embodiments, a sealant coating layer is coated onto the particles prior to applying the delayed release and/or enteric polymer coating layers. In still other embodiments, a sealant coating layer can be applied after applying the delayed release and/or enteric polymer coating layers.

The method includes removing the coating solvent, thereby forming delayed release beads 100 comprising a delayed release coating 120 formed on the particles of the solid dispersion. Pharmaceutical dosage forms can then be prepared from the delayed release beads 100, e.g. by compressing delayed release beads to into tablets or filling a capsule with the delayed release beads 100 using conventional methods. The pharmaceutical dosage forms can optionally contain additional excipients, as described herein. In one embodiment, delayed release beads 100 and optionally additional excipients, are compressed into tablets using an externally lubricated tablet press.

Several embodiments of the present disclosure relate to a method of preparing a pharmaceutical composition comprising an enteric tablet 200. In some embodiments of the method, the enteric tablet 200 includes a compressed tablet 210 and a polymeric coating 220, as described elsewhere herein. In some embodiments, the method includes forming an API blend by combining and mixing thoroughly the API with one or more pharmaceutically acceptable ingredients or excipients, which are described elsewhere herein. In several embodiments, the API includes histamine, a histamine salt, a histamine derivative, a salt of a histamine derivative, and any combination of the foregoing as described elsewhere herein. In certain embodiments, the API is a histamine salt. In several embodiments, the method includes forming a granulated API by granulating the API blend. In several embodiments, the method includes forming a granulated API by dry granulation of the API blend. In several embodiments, the method includes forming the compressed tablet 210 by processing the granulated API blend with unit operations suitable to form the compressed tablet 210. In several embodiments, the method includes forming the enteric tablet 200 by applying the polymeric coating 220 onto the compressed tablet 210 with suitable unit operations.

### Method of Treating Cancer

Embodiments of the present disclosure relate to a method for treating a cancer or a tumor. In several embodiments, the method is for treating a cancer. In other embodiments, the method is for treating a tumor. The method includes administering a pharmaceutical composition including delayed release beads 100 to a subject having the cancer or the tumor. As shown in FIG. 1, in some embodiments, a delayed release bead 100 includes a delayed release coating 120, a sealant coating layer 140, an amorphous layer 160, and an inert particle core 180. The amorphous layer 160 includes a solid dispersion of a therapeutically effective dosage of at least one active pharmaceutical ingredient (API). In several embodiments, the API includes histamine, a histamine salt, a histamine derivative, a salt of a histamine derivative, and any combination of the foregoing. In some embodiments, the API is histamine. In certain embodiments, the API is a histamine salt. In other embodiments, the API is a histamine derivative. In other embodiments, the API is a salt of a histamine derivative. The delayed release coating 120 includes a water insoluble polymer and an enteric polymer. In several embodiments, the delayed release coating 120 is insoluble at pH equal to or less than about 3.5. In some embodiments, the delayed release coating 120 is soluble at pH equal to or greater than about 6. The complete composition and the preparation of the delayed release beads 100 is included elsewhere herein.

In several embodiments, the method includes providing the therapeutically effective plasma concentration of the API measured in micromoles per liter in μmole/L equal to or greater than about 0.01, 0.05, 0.1, 0.15, 0.17, 0.18, 0.19, 0.2, 0.21, 0.22, 0.23, 0.24, 0.25, 0.3, 0.5, 1, 2, 5, 10, 20, 50, 100, or ranges including and/or spanning the aforementioned values. In several embodiments, the method includes providing the therapeutically effective plasma concentration of the API of at least about 0.2 μmole/L. In several embodiments, the method includes providing the therapeutically effective plasma concentration of the API for equal to or greater than about 0.1, 0.25, 0.5, 0.75, 1, 1.25, 1.5, 1.75, 2, 2.25, 2.5, 3, 3.5, 4, 4.5, 5, 6, 7, 8, 9, 10 h, or ranges including and/or spanning the aforementioned values. In several embodiments, the method includes providing the therapeutically effective plasma concentration of the API about 0.25 to about 5 h, or about 0.5 to about 3 h, or about 0.5 to about 1.25 h.

In several embodiments, the histamine salt comprises a histamine monocation, a histamine polycation, and any combination of the foregoing. In several embodiments, the histamine salt comprises a histamine dication. In several embodiments, the histamine salt comprises at least one anion selected from acetate, aspartate, citrate, formate, fumarate, halide, malate, nitrate, nitrite, phosphite, phosphate, succinate, sulfate, sulfite, tartrate, and any combination of the foregoing. In several embodiments, the histamine salt comprises histamine dihydrohalide or histamine phosphate. In several embodiments, the histamine salt comprises histamine dihydrochloride.

The term “histamine derivative,” as used herein, is a structural analog of histamine and/or a synthetically derived analog of histamine. In several embodiments, the histamine derivative comprises a C₁₋₆ alkyl histamine. In several embodiments, the histamine derivative comprises an N—(C₁₋₆ alkyl)histamine, a 4-(C₁₋₆ alkyl)histamine, a 4-(C₁₋₆ alkyl)histamine, and any combination of the foregoing. In several embodiments, the histamine derivative comprises N-methylhistamine or 4-methylhistamine. In several embodiments, the histamine derivative comprises N-methylhistamine.

In several embodiments, the salt of the histamine derivative comprises a C₁₋₆ alkyl histamine monocation, a C₁₋₆ alkyl histamine polycation and any combination of the foregoing. In several embodiments, the salt of the histamine derivative comprises a C₁₋₆ alkyl histamine dication. In several embodiments, the salt of the histamine derivative comprises an N—(C₁₋₆ alkyl)histamine dication, a 4-(C₁₋₆ alkyl)histamine dication, a 4-(C₁₋₆ alkyl)histamine dication, and any combination of the foregoing. In several embodiments, the salt of the histamine derivative comprises at least one anion selected from acetate, aspartate, citrate, formate, fumarate, halide, malate, nitrate, nitrite, phosphite, phosphate, succinate, sulfate, sulfite, tartrate, and any combination of the foregoing. In several embodiments, the salt of the histamine derivative comprises N-methylhistamine dihydrochloride or 4-methylhistamine dihydrochloride. In several embodiments, the salt of the histamine derivative comprises N-methylhistamine dihydrochloride.

In several embodiments, the method inhibits the production or release of intracellular hydrogen peroxide. In several embodiments, the method inhibits the production or release of intracellular hydrogen peroxide by monocytes. In an embodiment, the inhibition occurs when the API binds histamine H2 receptors. In several embodiments, the inhibition occurs when the API binds histamine H2 receptors selectively. In several embodiments, the method has an affinity for histamine H2 receptors that is higher compared to the affinity for any other histamine receptor. In several embodiments, the method has affinity for histamine H2 receptors that is higher compared to the affinity for a H1 receptor, a H3 receptor, a H4 receptor, and any combination of the foregoing.

The method includes providing a therapeutically effective dosage of at least one active pharmaceutical ingredient (API). In several embodiments, the therapeutically effective dosage of the API in units of milligrams (mg) is equal to or less than about: 0.01, 0.03, 0.07, 0.1, 0.2, 0.3, 0.4, 0.5, 0.6, 0.7, 0.8, 0.9, 1, 1.1, 1.3, 1.6, 2, 3, 5, 8, 10, 15, 18, 20, 30, 50, 100, or ranges including and/or spanning the aforementioned values. In several embodiments, the therapeutically effective dosage of the API is from about 0.1 mg to about 10 mg, or from about 0.5 mg to about 8 mg.

Some embodiments of the present disclosure relate to methods for administering the pharmaceutical compositions. In some embodiments, the method includes a route of administering the pharmaceutical compositions that is selected from the group consisting of oral, rectal, topical, aerosol, injection, parenteral, intramuscular, subcutaneous, intravenous, intramedullary, intrathecal, direct intraventricular, intraperitoneal, intranasal, intraocular, and any combination of the foregoing. In several embodiments, the method further comprises administering the pharmaceutical composition with an oral pathway. In several embodiments, the oral pathway includes administration in capsule, tablet, granule, pellet, spray, syrup, and any combination of the foregoing. In several embodiments, the method includes an oral dosage form selected from the capsules and tablets as described elsewhere herein.

In several embodiments, when the pharmaceutical composition is administered with the oral pathway, the API has a solubility equal to or greater than about: 40%, 50%, 60%, 70%, 75%, 80%, 85%, 87%, 89%, 90%, 91%, 92%, 93%, 94%, 95%, 96%, 97%, 98%, 99%, 99.5%, or ranges including and/or spanning the aforementioned values. In several embodiments, when the pharmaceutical composition is administered with the oral pathway, the API has a solubility from about 70% to 99.5%, or about 85% to 99%, or about 95% to 99%. In several embodiments, when the pharmaceutical composition is administered with the oral pathway, the pharmaceutical composition has a solubility equal to or greater than about: 40%, 50%, 60%, 70%, 75%, 80%, 85%, 87%, 89%, 90%, 91%, 92%, 93%, 94%, 95%. 96%, 97%, 98%, 99%, 99.5%, or ranges including and/or spanning the aforementioned values. In several embodiments, when the pharmaceutical composition is administered with the oral pathway, the solubility of histamine dihydrochloride is equal to or greater than about: 40%, 50%, 60%, 70%, 75%, 80%, 85%, 87%, 89%, 90%, 91%, 92%, 93%, 94%, 95%, 96%, 97%, 98%, 99%, 99.5%, or ranges including and/or spanning the aforementioned values. In several embodiments, when the pharmaceutical composition is administered with the oral pathway, the solubility of the API, the pharmaceutical composition, and/or histamine dihydrochloride is measured at a pH of about 5, 6, 6.6, 6.8, 7, 7.2, 7.4, 7.6, 8, 8.2, 8.4 or ranges including and/or spanning the aforementioned values. In several embodiments, when the pharmaceutical composition is administered with the oral pathway, the solubility of the API, the pharmaceutical composition, and/or histamine dihydrochloride is measured at a pH from about 7 to 7.8 or about 7.2 to 7.6.

In several embodiments, when the pharmaceutical composition is administered with the oral pathway, the API has an apparent permeability expressed in μcm/s equal to or greater than about 1, 3, 5, 13, 15, 16, 17, 18, 19, 20, 21, 22, 23, 24, 28, 30, 40, or ranges including and/or spanning the aforementioned values. In several embodiments, when the pharmaceutical composition is administered with the oral pathway, the API has an apparent permeability from about 1 to 40 μcm/s, about 12 to 24 μcm/s, about 16 to 20 μcm/s. In several embodiments, when the pharmaceutical composition is administered with the oral pathway, the pharmaceutical composition has an apparent permeability expressed in μcm/s equal to or greater than about 1, 3, 5, 13, 15, 16, 17, 18, 19, 20, 21, 22, 23, 24, 28, 30, 40, or ranges including and/or spanning the aforementioned values. In several embodiments, when the pharmaceutical composition is administered with the oral pathway, the pharmaceutical composition has an apparent permeability from about 1 to 40 μcm/s, about 12 to 24 μcm/s, about 16 to 20 μcm/s. In several embodiments, when the pharmaceutical composition is administered with the oral pathway, histamine dihydrochloride has an apparent permeability expressed in μcm/s equal to or greater than about 1, 3, 5, 13, 15, 16, 17, 18, 19, 20, 21, 22, 23, 24, 28, 30, 40, or ranges including and/or spanning the aforementioned values. In several embodiments, when the pharmaceutical composition is administered with the oral pathway, histamine dihydrochloride has an apparent permeability from about 1 to 40 μcm/s, about 12 to 24 μcm/s, about 16 to 20 μcm/s. In several embodiments, when the pharmaceutical composition is administered with the oral pathway, the apparent permeability of the API, the pharmaceutical composition, and/or histamine dihydrochloride is measured across the small intestine. In several embodiments, when the pharmaceutical composition is administered with the oral pathway, the apparent permeability of the API, the pharmaceutical composition, and/or histamine dihydrochloride is measured at a pH of about 5, 6, 6.6, 6.8, 7, 7.2, 7.4, 7.6, 8, 8.2, 8.4 or ranges including and/or spanning the aforementioned values. In several embodiments, when the pharmaceutical composition is administered with the oral pathway, the apparent permeability of the API, the pharmaceutical composition, and/or histamine dihydrochloride is measured at a pH from about 7 to 7.8 or about 7.2 to 7.6.

In several embodiments, when the pharmaceutical composition is administered with the oral pathway, the API is absorbed in the small intestine in an amount equal to or greater than about: 40%, 50%, 60%, 70%, 75%, 80%, 81%, 82%, 83%, 84%, 85%, 86%, 87%, 88%, 89%, 90%, 95%, 98%, 99%, 99.5% of the therapeutically effective dosage, or ranges including and/or spanning the aforementioned values. In several embodiments, when the pharmaceutical composition is administered with the oral pathway, the API is absorbed in the small intestine in an amount from about 40% to 99.5%, or about 75% to 99%, or about 80% to 90%. In several embodiments, when the pharmaceutical composition is administered with the oral pathway, histamine dihydrochloride is absorbed in the small intestine in an amount equal to or greater than about: 40%, 50%, 60%, 70%, 75%, 80%, 81%, 82%, 83%, 84%, 85%, 86%, 87%, 88%, 89%, 90%, 95%, 98%, 99%, 99.5% of the therapeutically effective dosage, or ranges including and/or spanning the aforementioned values. In several embodiments, when the pharmaceutical composition is administered with the oral pathway, histamine dihydrochloride is absorbed in the small intestine in an amount from about 40% to 99.5%, or about 75% to 99%, or about 80% to 90%.

In several embodiments, when the pharmaceutical composition is administered with the oral pathway, the bioavailability of the API is equal to or greater than about: 40%, 50%, 60%, 70%, 75%, 80%, 81%, 82%, 83%, 84%, 85%, 86%, 87%, 88%, 89%, 90%, 95%, 98%, 99%, 99.5% of the therapeutically effective dosage, or ranges including and/or spanning the aforementioned values. In several embodiments, when the pharmaceutical composition is administered with the oral pathway, the bioavailability of the API is from about 40% to 99.5%, or about 75% to 99%, or about 80% to 90%. In several embodiments, when the pharmaceutical composition is administered with the oral pathway, the bioavailability of the pharmaceutical composition is equal to or greater than about: 40%, 50%, 60%, 70%, 75%, 80%, 81%, 82%, 83%, 84%, 85%, 86%, 87%, 88%, 89%, 90%, 95%, 98%, 99%, 99.5% of the therapeutically effective dosage, or ranges including and/or spanning the aforementioned values. In several embodiments, when the pharmaceutical composition is administered with the oral pathway, the bioavailability of the pharmaceutical composition is from about 40% to 99.5%, or about 75% to 99%, or about 80% to 90%. In several embodiments, when the pharmaceutical composition is administered with the oral pathway, the bioavailability of histamine dihydrochloride is equal to or greater than about: 40%, 50%, 60%, 70%, 75%, 80%, 81%, 82%, 83%, 84%, 85%, 86%, 87%, 88%, 89%, 90%, 95%, 98%, 99%, 99.5% of the therapeutically effective dosage, or ranges including and/or spanning the aforementioned values. In several embodiments, when the pharmaceutical composition is administered with the oral pathway, the bioavailability of histamine dihydrochloride is from about 40% to 99.5%, or about 75% to 99%, or about 80% to 90%.

In several embodiments, the method includes a lag time as described elsewhere herein. In several embodiments, the lag time is equal to or greater than about 0.1, 0.25, 0.5, 0.75, 1, 1.25, 1.5, 1.75, 2, 2.25, 2.5, 3, 3.5, 4, 4.5, 5, 6, 7, 8, 9, 10 h, or ranges including and/or spanning the aforementioned values. In several embodiments, the lag time is from 0.1 to about 10 h, or about 0.25 to about 3 h, or about 0.5 to about 1.25 h, or about 0.5 to about 1 h. The lag time imparts several beneficial properties to the method of the present disclosure: the method prevents the release of the API in the stomach; the method avoids stimulating histamine H2 receptors in the stomach; the method avoids stimulating the parietal cells in the stomach; and the method avoids stimulating excess release of stomach acid. In several embodiments, the method provides an oral pathway of administration that is safe, or not otherwise harmful, to the gastro-intestinal (GI) tract.

Natural killer (NK) cells have a critical role in the tumor microenvironment (TME) associated with cancer development and progression. Within the TME, cells from adaptive and innate immune system, including NK cells, are one of the most abundant components. The dynamic interactions between immune and cancer cells create complex molecular mechanisms that can deactivate NK cells and sustain tumor growth. In several embodiments, the method avoids inactivation of natural killer cells in the tumor micro-environment. In several embodiments, the method increases activation of natural killer cells in the tumor micro-environment.

In some embodiments, the treatment outcome of second form of cancer therapy is improved when the pharmaceutical composition is administered concurrently with the second form of cancer therapy. In several embodiments, the method further comprises administering at least one other cancer therapy. In some embodiments, the other cancer therapy is selected from the group consisting of surgery, radiation, immunotherapy, chemotherapy, and any combination of the foregoing. In certain embodiments, the other cancer therapy is immunotherapy. In some embodiments, the other cancer therapy includes treatment with at least one cytokine. In other embodiments, the other cancer therapy includes treatment with Interleukin-2. In some embodiments, the includes cancer therapy includes administration of an agent which enhances the humoral immune response.

In some embodiments, the cancer is a blood cancer selected from the group consisting of leukemia, lymphoma, myeloma, and any combination of the foregoing. In other embodiments, the cancer or the tumor is a solid cancer selected from the group consisting of breast, lung, prostate, colorectal, melanoma and bladder. In several embodiments, the cancer or the tumor is selected from the group consisting of a leukemia, a myeloma, a bone marrow cancer, a Hodgkin's lymphoma, a Non-Hodgkin's lymphoma, a follicular lymphoma, a lymphoid malignancy of T-cell or B-cell origin, a bladder cancer, a brain cancer, a breast cancer, a cervical cancer, a colorectal cancer, an esophageal cancer, a hepatocellular cancer, a melanoma, a head and neck cancer (including oral cancer), an ovarian cancer, a small cell lung cancer, a non-small cell lung cancer, a prostate cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma, a sarcoma, a carcinoma, an Ewings's tumor, a Wilm's tumor, and any combination of the foregoing.

In several embodiments, the cancer is a leukemia. In other embodiments, the cancer is a leukemia selected from the group consisting of an acute myeloid leukemia, a lymphoblastic leukemia, a myelogenous leukemia, a chronic lymphocytic leukemia, a chronic myeloid leukemia, and any combination of the foregoing.

In certain embodiments, the method includes administering the pharmaceutical composition in combination with a cytokine to a subject in a first remission from leukemia. In other embodiments, the method includes administering the pharmaceutical composition in combination with a cytokine to a subject in a subsequent remission from leukemia after relapse. In an embodiment, the cytokine is Interleukin-2.

Some embodiments relate to a method for treating a cancer that includes contacting a malignant growth with the pharmaceutical composition of the present disclosure. Other embodiments relate to a method for treating a cancer that includes contacting a tumor with the pharmaceutical composition of the present disclosure. Some embodiments relate to a method for inhibiting replication of a malignant growth that includes contacting a malignant growth with the pharmaceutical composition of the present disclosure. Other embodiments relate to a method for inhibiting replication of a tumor that includes contacting a tumor with the pharmaceutical composition of the present disclosure.

Some embodiments relate to a method for preventing metastasis of a cancer or tumor. In some embodiments, the method includes oral administration of the pharmaceutical composition of the present disclosure.

Some embodiments relate to a pharmaceutical composition as described herein for use in treating a cancer or a tumor. Some embodiments relate to a pharmaceutical composition as described herein for use in treating a malignant growth. Some embodiments relate to a pharmaceutical composition as described herein for use in inhibiting replication of a malignant growth or a tumor. In several embodiments, the pharmaceutical composition is formulated as an oral dosage.

Some embodiments relate to the use of a pharmaceutical composition as described herein for treating a cancer or a tumor. Some embodiments relate to the use of a pharmaceutical composition as described herein for treating a malignant growth. Some embodiments relate to the use of a pharmaceutical composition as described herein for inhibiting replication of a malignant growth or a tumor. In several embodiments, the pharmaceutical composition is formulated as an oral dosage.

Some embodiments relate to the use of an effective amount of the pharmaceutical composition of the present disclosure in the manufacture of a medicament for treating a cancer or a tumor. Other embodiments relate to the use of an effective amount of the pharmaceutical composition of the present disclosure in the manufacture of a medicament for treating a malignant growth or a tumor. Certain embodiments relate to the use of an effective amount of the pharmaceutical composition of the present disclosure in the manufacture of a medicament for inhibiting replication of a malignant growth or a tumor. In several embodiments, the pharmaceutical composition is formulated as an oral dosage.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The above description discloses several methods and materials of the present disclosure. This disclosure is susceptible to modifications in the methods and materials, as well as alterations in the fabrication methods and equipment. Such modifications will become apparent to those skilled in the art from a consideration of this disclosure or practice of the disclosure disclosed herein. Consequently, it is not intended that this disclosure be limited to the specific embodiments disclosed herein, but that it covers all modifications and alternatives coming within the true scope and spirit of the disclosure.

### EXAMPLES

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### Example 1

### Histamine Dihydrochloride Beads

This is a prophetic example. A polymer solution is prepared by adding slowly Kollidon VA 64 (800 g) to a 72.5/22.5/5 mixture of 95% ethanol/acetone/water (4930 g/1530 g/340 g) with vigorous stirring until dissolved, and then histamine dihydrochloride (400 g) is added slowly until dissolved. A Glatt GPCG 3 equipped with a 7″ bottom spray/8″ column height Wurster insert, 20 mm partition gap, air-distribution plate B (250 μm screen), 1.0 mm nozzle port, atomization air pressure of 1.5 bar, and 3.2 mm inner diameter tubing, is charged with 2584 g of 25-30 mesh Sugar Spheres. About 40 g of talc is homogenized into the polymer solution to minimize static build-up. The histamine dihydrochloride polymer solution, at a solids content of 15% by weight, is sprayed onto the sugar spheres at a spray rate of 8-17 g/min and outlet flap at ˜60-80% (air velocity: ˜85-115 m³/h) while maintaining the product temperature at about 36-40° C. The resulting histamine dihydrochloride beads (batch size: 3724 g) are dried in the Glatt unit at 40° C. for about 45 min to minimize the residual solvent level in the product. A 98.5% yield of uscable beads (600-1200 μm) is obtained.

2800 g of histamine dihydrochloride beads are provided with coating weight of 2% (i.e., weight of the coating relative to the weight of uncoated beads) protective seal-coat of OPADRY Clear (at 8% by weight solids; product temperature: 37-41° C.; spray rate: 5-12 g/min), are further dried at 40° C. in the Glatt unit for about 45 min to drive off residual solvent/moisture. The measured potency is 9.81% (% histamine dihydrochloride) compared to the target potency of 10% histamine dihydrochloride.

### Example 2

### Histamine Dihydrochloride Delayed Release Beads

This is a prophetic example. A delayed release coating solution of Eudragit RL/Eudragit L/TEC/talc at a solids content of 10% solids is prepared by first slowly adding Eudragit RL polymer to a solvent mixture of 45/55 acetone/ethanol to achieve a clear solution while stirring. Next, the Eudragit L polymer and then the plasticizer (triethylcitrate or “TEC”) are slowly added and allowed to dissolve in the solution. Talc is separately suspended in the solvent mixture using an Ultraturrex homogenizer before being added to the dissolved polymers and plasticizer. A Glatt GPCG 1 equipped with a 4″ bottom spray Wurster insert, 20 mm partition gap, air-distribution plate B (250 μm screen), 1.0 mm nozzle port, atomization air pressure of 1.5 bar, and 3.2 mm inner-diameter tubing, and a TI 65P dedicated filter bag, is used to apply the delayed release coating solution to the histamine dihydrochloride beads (700 g) having a nominal drug loading of 10%, prepared as described in Example 1. The delayed release coating solution (10% solids content), is sprayed at a spray rate of 4-11 g/min, outlet flap at ˜20-30% (air velocity: ˜2.0-2.5 m/s), and at a product temperature of 35-38° C. Samples are pulled to provide coatings with coating weights of 5%, 10%, 15%, and 20% by weight. The coated beads are dried in the Glatt at 40° C. for 45 minutes to drive off excess residual solvents. The dried histamine dihydrochloride delayed release beads are sieved to discard any doubles (i.e., two or more beads adhered together by the delayed release coating), if formed.

### Example 3

### Histamine Dihydrochloride—Fumaric Acid Delayed Release Beads

This is a prophetic example. Histamine dihydrochloride-fumaric acid beads are prepared by layering a 1/2/1 weight ratio solution of histamine dihydrochloride/Kollidon VA 64/fumaric acid dissolved in ethanol/acetone/water, onto 25-30 mesh sugar spheres in a Glatt GPCG 3, and a nominal histamine dihydrochloride loading of 10% by weight, using the procedure described in Example 1. The histamine dihydrochloride-fumaric acid beads (700 g) are coated with a 35/50/10/5 Eudragit RL/Eudragit L/TEC/talc TPR coating at a coating weight of up to 30% (samples are pulled at a coating level of 10%, 15%, 20%, 25%, and 30%), using the procedure described in Example 2 to produce histamine dihydrochloride-fumaric acid delayed release beads.

### Example 4

### N-Methyl Histamine Beads

This is a prophetic example. N-methyl histamine beads are prepared with the procedure described in Example 1 by using 400 g N-methyl histamine in place of histamine dihydrochloride. The measured potency is 9.81% (% N-methyl histamine) compared to the target potency of 10% N-methyl histamine.

### Example 5

### N-Methyl Histamine Delayed Release Beads

This is a prophetic example. N-methyl histamine delayed release beads are prepared with the procedure described in Example 2 by using 700 g N-methyl histamine beads prepared in Example 4 in place of histamine dihydrochloride beads. Samples are pulled to provide coatings with coating weights of 5%, 10%, 15%, and 20% by weight.

### Example 6

### N-Methyl Histamine—Fumaric Acid Delayed Release Beads

This is a prophetic example. N-methyl histamine-fumaric acid beads are prepared with the procedure described in Example 3 by using N-methyl histamine in place of histamine dihydrochloride. The N-methyl histamine-fumaric acid beads (700 g) are coated with a 35/50/10/5 Eudragit RL/Eudragit L/TEC/talc TPR coating at a coating weight of up to 30% (samples are pulled at a coating level of 10%, 15%, 20%, 25%, and 30%) to produce N-methyl histamine dihydrochloride-fumaric acid delayed release beads.

### Example 7

### Effects on NK Cell Function

This is a prophetic example. The effect of the pharmaceutical composition comprising the delayed release beads prepared as described in Example 2 on NK cell function is evaluated using an in vivo mouse model assay. Mice are injected with a solution of the pharmaceutical composition, ranitidine or a control solution. Twenty-four hours later they are injected intravenously with ⁵¹Cr-labeled YAC-1 lymphoma cells which form tumor emboli in the lungs and which are extremely sensitive to lysis by NK cells. Four hours after the YAC-1 injection the animals are sacrificed and the radioactivity present in their lungs is measured. The levels of radioactivity in lungs has been shown to be inversely proportional to NK cell function in that the greater the lysis by NK cells of the target YAC-1 cells the less radioactivity is shown in the lungs. Treatment with the pharmaceutical composition reduces the amount of lung radioactivity by up to ⅔ when compared to the control group. Treatment with the H2 antagonist, ranitidine, results in a tripling of the level of radioactivity.

### Example 8

### Pharmacodynamic Studies

This is a prophetic example. The pharmaceutical composition comprising the delayed release beads prepared as described in Example 2 is administered in various animal tumor models for the pharmacodynamic studies shown in Table 1. The effect of the pharmaceutical composition on NK sensitive cells and H2 receptor involvement described in Example 7 is shown to be most effective when the pharmaceutical composition is administered 2-6 hours prior to cell inoculation.

TABLE 1

| Tumour model | Species | Dosage | Effects |
|---|---|---|---|
| Fibrosarcoma | Mouse | 6 mg/day i.p., | Reduced tumour |
| | | daily | growth and |
| | | | improved survival |
| Chemically induced | Rat | 4 mg/day s.c., | Reduced glandular |
| gut tumours | | daily | tumour in rats |
| Ascitis sarcoma | Rat | 0.005 μg/day i.p. | Improved survival |
| Chemically | Rat | 0.1 mg/kg/day | Reduced tumour |
| induced mammary | | s.c., daily | incidence and |
| carcinoma | | | growth |
| Melanoma | Mouse | 2.5-250 mg/kg | Reduced pulmonary |
| | | i.v., single dose | metastasis |
| YAC-1 | Mouse | 125 mg/kg i.v., | Reduced YAC-1 lung |
| lymphoma | | single dose | tumour emboli |
| YAC-1 | Mouse | 125 mg/kg i.v., | Improved NK cell |
| lymphoma | | single dose | clearance of YAC-1 |
| | | | tumour cells |
| Colorectal | Mouse | 1 mg/kg/day i.p. | Reduced growth |
| carcinomas | | daily | of human tumours |
| | | | transplanted to the |
| | | | subrenal capsule |
| Syngeneic | Rat | 0.5 mg/kg/day | Reduced growth rate |
| colorectal | | s.c. daily | of transplanted |
| adenocarcinoma | | | liver tumours in rats |
| Leydig cell | Rat | 0.5 mg/kg/day | Reduced growth rate |
| Sarcoma | | s.c. daily | of transplanted |
| | | | liver tumours in rats |
| Colorectal cancer | Mouse | Local adminis- | Enhanced tumour |
| | | tration via | growth |
| | | osmotic pump | |
| Leukaemia | Mouse | 5 μg/day; i.p. | Slightly increased |
| | | daily for 5 days | survival in mice |
| | | | inoculated with |
| | | | murine B-cell |
| | | | leukaemia |

Note:
i.p. = intraperitoneal;
i.v. = intravenous;
s.c. = subcutaneous

### Example 9

Combination Therapy with IL-2 in Animal Models

This is a prophetic example. Interleukin-2 is administered in combination with the pharmaceutical composition comprising the delayed release beads prepared as described in Example 2 in various animal tumor models for the studies shown in Table 1. IL-2 only weakly activates NK cells or T cells in an environment of oxidative stress. The pharmaceutical composition inhibits production of reactive oxygen species and protects NK cells and T cells from down-regulation and apoptosis thereby improving the IL-2-induced activation of T cells and NK cells in tumors. The combined treatment effectively protects mice against B16 melanoma lung metastases.

TABLE 2

| Tumour model | Species | Effects |
|---|---|---|
| B16 melanoma | Mouse | Potentiate reduction of metastatic lung |
| | | tumours by IL-2 |
| YAC-1 lymphoma | Mouse | Enhance NK cell mediated clearance of |
| | | YAC-1 cells from lungs by IL-2 |
| Dunning prostate | Rat | Potentiate anti-tumour properties of IL-2 |
| adenocarcinoma | | |
| Intra-cerebral | Rat | Reduce growth of established intracerebral |
| glioma | | tumours by 50% |

### Example 10

### Treatment of Leukemia

Interleukin-2 administered in combination with the pharmaceutical composition comprising the delayed release beads prepared as described in Example 2 is an immunotherapy which aims to induce immune-mediated destruction of residual myeloid leukemia cells and thereby to prevent relapse of leukemia. The role the histidine hydrochloride delayed release beads is to protect lymphocytes, in particular NK cells and T cells, which are responsible for the immune-mediated destruction of residual leukemic cells. The role of IL-2 is to promote the functions of NK cells and T cells by activating the anti-leukemic properties of these cells and by expanding these cell populations by inducing cell cycle proliferation. The mechanism by which the histidine hydrochloride delayed release beads improves the anti-leukemic function of lymphocytes in AML is considered to be by inhibition of reactive oxygen species (ROS or ‘oxygen free radicals’), which are synthesized by monocytes/macrophages and granulocytes. ROS are known to limit the anti-leukemic effects of lymphocyte activators such as IL-2, by triggering dysfunction and death by apoptosis in NK cells and T cells. The histidine hydrochloride delayed release beads inhibit NAPDH oxidase which initiates the formation and release of ROS from phagocytes. By inhibiting oxidase function and reducing ROS production, the histidine hydrochloride delayed release beads protect IL-2-activated NK cells and T cells from oxygen free radical-induced inhibition and apoptosis.

This is a prophetic example. Recombinant Interleukin-2 (aldesleukin, IL-2) is administered at a dosage of 16,400 IU/kg (1 μg/kg) twice daily as a subcutaneous injection. The pharmaceutical composition comprising the delayed release beads prepared as described in Example 2 is orally administered in a dosage of 5 mg from 1 to 3 minutes after each injection of IL-2. The dual therapy is administered for 10 treatment cycles: each cycle consists of a treatment period of 21 days (3 weeks) followed by a three-week or six-week treatment-free period. For cycles 1-3, each cycle consists of 3 weeks of treatment, followed by a 3-week treatment free period. For cycles 4-10, each cycle consists of 3 weeks of treatment, followed by a 6-week treatment free period. The 10 treatment cycles are completed over an 81-week period.

In a first study, the participants are 261 individual patients in a first remission from leukemia. The study participants are split into a group of experimental patients and control patients. The experimental patients are administered the dual therapy for 10 treatment cycles over 81 weeks. The control group receives a placebo containing magnesium stearate in the same treatment cycle and duration. The median duration of leukemia-free survival is 291 days (9.7 months) in the control patients and 450 days (15 months) in the experimental patients.

In a second study, the participants are 59 individual patients in a subsequent remission from leukemia after relapse. The study participants are split into a group of experimental patients and control patients. The experimental patients are administered the dual therapy for 10 treatment cycles over 81 weeks. The control group receives a placebo containing magnesium stearate in the same treatment cycle and duration. The number of in the experimental patients remaining leukemia-free for 3 years is 26% in the control patients and 40% in the experimental patients.

### Example 11

### Human Small Intestinal Absorption & Permeability Model

This example describes a comparison of performance in predicting human drug absorption data using drug permeability data from MATTEK EPIINTESTINAL full-thickness tissue culture and Caco-2 cells. Caco-2 cell-based cultures are used routinely for drug absorption modelling studies even though they form a non-physiological barrier due to paracellular junctions that are tighter and, therefore, less permeable than the native intestinal epithelium. The MATTEK EPIINTESTINAL full-thickness tissue culture is an organotypic 3D human small intestinal (SMI) microtissue model which more accurately mimics native human small intestine tissue compared to Caco-2 cell-based cultures. The polarity and defined structural geometry of the MATTEK EPIINTESTINAL SMI microtissues allows for apical (“luminal side”) application of drugs to study lumen-to-bloodstream drug transport. The MATTEK EPIINTESTINAL SMI microtissues express intestinal drug metabolizing enzymes and drug transporters at levels similar to those of the in vivo intestinal tissue. (Ayehunie, et al. Pharmaceutical Research, 2018, 1-18).

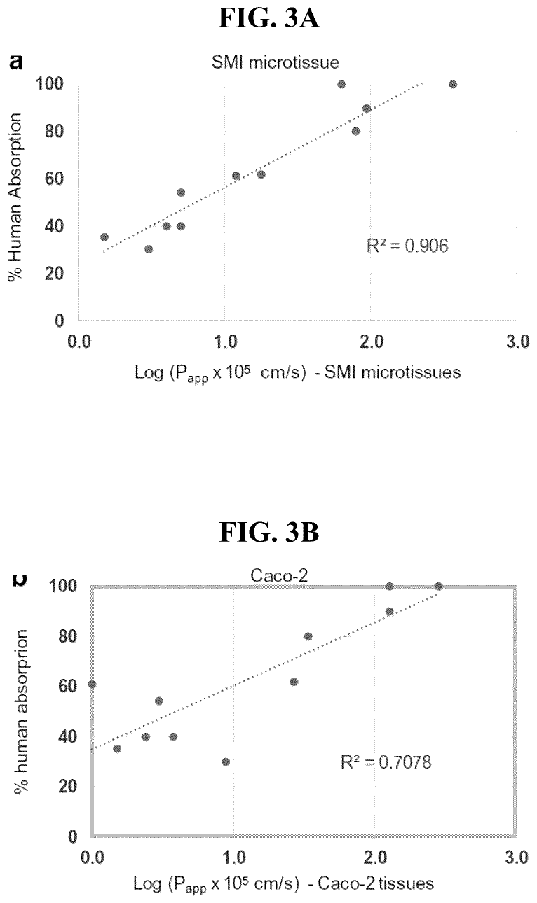

The comparative studies included 11 model drug compounds having low, moderate, and high absorption in humans, as recommended by FDA. Triplicate tissues were exposed to each model drug (n=11) for 2 h. Average apparent permeability (Papp) values from two independent lots of MATTEK EPIINTESTINAL SMI microtissues were used. The results of the comparative studies indicated that the coefficient of correlation (R²) between the Papp and human absorption data was 0.906 for the MATTEK EPIINTESTINAL SMI microtissues and 0.708 for Caco-2 monolayer cells, respectively, as shown in FIG. 3A and FIG. 3B. The improved performance of the MATTEK EPIINTESTINAL SMI microtissues compared to Caco-2 cells can be attributed to the superior combination of the structure, barrier properties, and expression of key transporters and enzymes of the MATTEK EPIINTESTINAL SMI microtissues. (Ayehunie, et al. Pharmaceutical Research, 2018, 1-18).

### Example 12

### Permeability of Histamine Dihydrochloride

The unidirectional permeability of a pharmaceutical composition comprising histamine dihydrochloride was determined by measuring cumulative receiver concentration across MATTEK EPIINTESTINAL SMI microtissues. Histamine dihydrochloride (Cat. #H436500, Lot 5-SCC-58-1) was procured from Toronto Research Chemicals (Toronto, Ontario, Canada). The reference compound histamine-α,α,β,β-d₄ dihydrochloride (Cat. #762962, Lot MBBC8722), atenolol (Cat. #A7655) and caffeine (Cat. #C0750) were obtained from MilliporeSigma (St. Louis, MO, USA). Phosphate-buffered saline (PBS, Cat. #10010 from ThermoFisher Scientific, Waltham, MA, USA) was adjusted to pH 7.4 and used as the receiver buffer. MATTEK EPIINTESTINAL SMI microtissue cultures with surface area of 0.6 cm² (Cat. #SMI-100-FT, lot #33479) were procured from MatTek Corporation (Ashland, MA, USA). Tissues were received and stored at 4° C. until use. The tissue inserts were transferred to plates containing MATTEK SMI-100 culture medium using sterile technique and placed in a humidified incubator (37±1° C., 5% CO₂) overnight for recovery prior to use, according to MATTEK protocols.

The pharmaceutical composition was prepared from histamine dihydrochloride (10 mg/mL or 89969 μM) in PBS, pH 7.4. Reference A was prepared from caffeine (50 μM) and atenolol (100 μM) co-dosed in PBS, pH 7.4. The control was PBS, pH 7.4 buffer for baseline endogenous histamine. Reference B was histamine α,α,β,β-d4 dihydrochloride (1 mg/mL) in PBS, pH 7.4. Reference A was a 300 μL dose added to the apical side of the tissue. A pH of 7.4 was chosen in order to simulate the intraluminal pH of the small intestine in humans, which gradually increases from 6.6 in the proximal small intestine to about 7.5 in the terminal ileum. (Evans, et al. Gut, 1988, 1035-1041). In each trial, a 100 μL sample was immediately collected as the 0-minute donor sample and was not replenished. The pharmaceutical composition, the control, and Reference B were dosed with 200 μL, and 10 μL donor samples and collected at each time point (0, 15, 30, 60, and 120 minutes) without replacement. Six replicates of each treatment were conducted. All dosed MATTEK EPIINTESTINAL inserts were transferred to a 12-well plate containing 5 mL of PBS, pH 7.4 to initiate the assay. The assay plates were transferred to a humidified incubator (37±1° C., 5% CO₂) between sampling time points. At each time point, receiver samples (200 μL) were collected for measurement of the concentration of the compound that had collected in the receiver and 200 μL of blank pre-warmed PBS, pH 7.4 was added back to assay plates. Upon completion of the study, all samples were stored at −80° C. until analysis by LC-MS/MS. The control and Reference B donor samples were diluted 10-fold with PBS, then mixed with an equal volume of deionized water containing 0.2 μM caffeine-d9 and atenolol-d7. The pharmaceutical composition receiver and donor samples were diluted 10-fold and 200-fold, respectively, with acetonitrile (MeCN):PBS, 9:1, v:v, then mixed with an equal volume of deionized water containing 0.02 μg/mL histamine-α,α,β,β-d₄ dihydrochloride before analysis by LC-MS/MS.

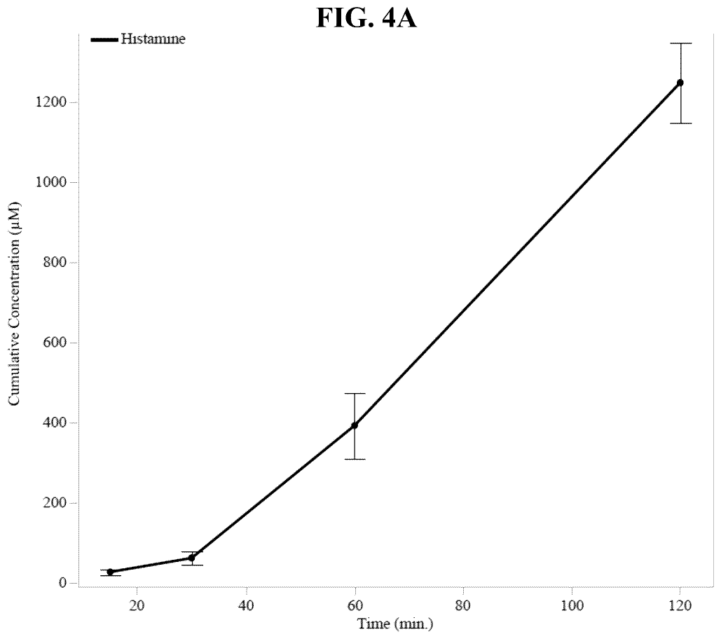

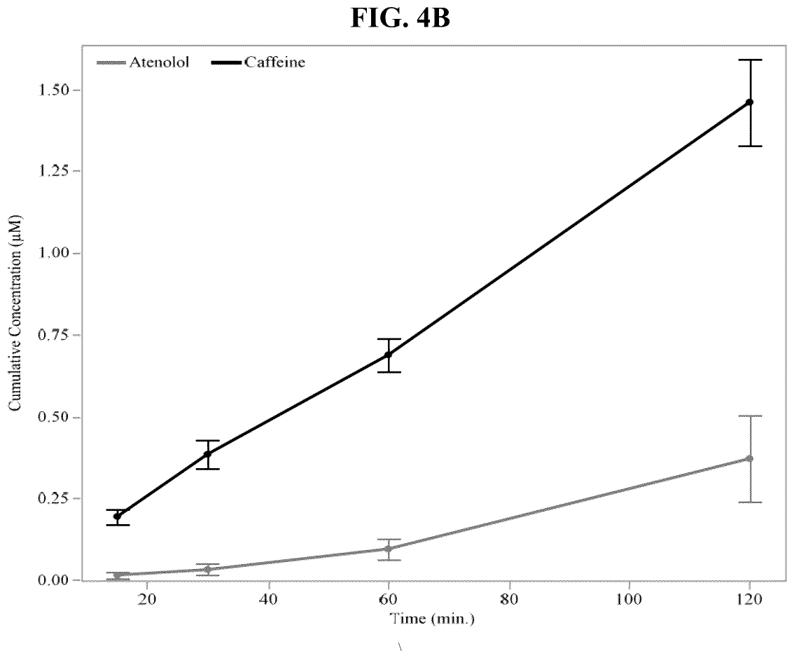

Results for the mean cumulative receiver concentrations across the MATTEK EPIINTESTINAL SMI microtissue cultures measured at each time point for histamine dihydrochloride (the pharmaceutical composition), and co-dosed caffeine and atenolol (Reference A), are shown in Table 3. The mean cumulative receiver concentrations plotted over time for histamine dihydrochloride and the reference compounds are shown in FIG. 4A and FIG. 4B, respectively. The difference in concentration of at least 3-fold between caffeine (highly permeable) and atenolol (moderately permeable) observed in FIG. 4B indicates the robust barrier function of the MATTEK EPIINTESTINAL SMI microtissues

TABLE 3

| | | Cumulative Receiver Concentration | | | |
|---|---|---|---|---|---|
| | | (μM) | | | |
| | | 15 min | 30 min | 60 min | 120 min |
| histamine | Mean (n = 6) | 27.2 | 62.2 | 392 | 1247 |
| dihydrochloride | SD | 7.6 | 16.6 | 83 | 100 |
| | CV (%) | 27.8 | 26.7 | 21.1 | 8.03 |
| atenolol | Mean (n = 6) | 0.0138 | 0.0310 | 0.0937 | 0.370 |
| (co-dosed with | SD | 0.0096 | 0.0173 | 0.0321 | 0.133 |
| caffeine) | CV (%) | 69.9 | 55.9 | 34.2 | 35.9 |
| caffeine | Mean (n = 6) | 0.193 | 0.383 | 0.687 | 1.46 |
| (co-dosed with | SD | 0.023 | 0.044 | 0.051 | 0.13 |
| atenolol) | CV (%) | 11.9 | 11.6 | 7.47 | 9.02 |

The apparent permeability (Papp), flux, and solution recovery values were calculated from the cumulative receiver concentrations of histamine dihydrochloride and the co-dosed reference compounds. The rank order of Papp values was determined to be caffeine (33.3±4.0)>histamine dihydrochloride (18.7±1.5)>atenolol (4.84±1.67), reported in μcm/s, as shown in Table 4. The reported Papp values were normalized for differences in concentration of the assay solutions: caffeine (50 μM), histamine dihydrochloride (89969 μM), atenolol (100 μM). The difference in the Papp between caffeine and atenolol of more than 6-fold further confirms the robust barrier function of the MATTEK EPIINTESTINAL SMI microtissue culture as discussed above regarding FIG. 4B.

The result of a solution recovery for histamine dihydrochloride of 93.6% indicates that histamine remains in solution nearly quantitatively after the incubation period. The endogenous levels of histamine in the control samples were below the limit of quantification (0.0270 μM).

TABLE 4

| | | caffeine | atenolol |
|---|---|---|---|
| | histamine | (co-dosed with | (co-dosed with |
| | dihydrochloride | atenolol) | caffeine) |
| Papp (μcm/s) | 18.7 ± 1.5 | 33.3 ± 4.0 | 4.84 ± 1.67 |
| Flux (μmol/ | 101 ± 8 | 0.0001 ± 0.000012 | 0.000029 ± 0.0001 |
| min/cm²) | | | |
| Recovery | 93.6 ± 2.2 | 101 ± 3 | 89.1 ± 5.2 |
| (%) | | | |

### Example 13

### Bioavailability of Histamine Dihydrochloride

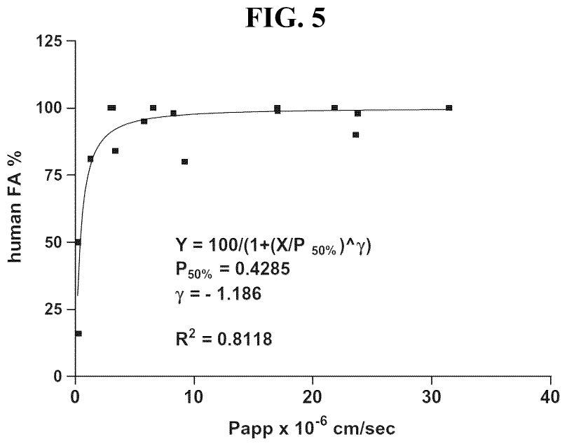

Previous studies of the drug permeability data in Caco-2 cells for predicting human drug absorption data plotted apparent permeability (Papp) values against the drug fraction absorbed in humans expressed as a percentage of the administered dose (FA). The studies resulted in a regression analysis showing that compounds having Papp of more than 10 μcm/s were absorbed in the small intestine in amounts greater than 85% of the administered dose, as shown in FIG. 5. (Turco, et al. Toxicology in Vitro, 2010, 13-20). Therefore, the Papp of 18.7±1.5 cm/s for histamine dihydrochloride determined in Example 12 indicates that when the pharmaceutical composition including histamine dihydrochloride is administered orally, histamine dihydrochloride will be absorbed in the small intestine in an amount greater than 85% of the amount of the administered dosage.

### Example 14

### Data Analysis and Calculations

The cumulative receiver concentration of each compound at each time point (C_{c}) was calculated as the sum of the measured concentration at that time point (Cₘ) and 0.2/5 of the measured concentrations at the previous time points (a 0.2 mL receiver sample out of the 5 mL total receiver volume was withdrawn and replaced at each time point, except for the final time point which was not replaced).

For receiver samples at 15, 30, 60, and 120 minutes: C_{c15}=Cₘ₁₅; C_{c30}=Cₘ₃₀+Cₘ₁₅×0.2/5; C_{c60}=Cₘ₆₀+(Cₘ₁₅+Cₘ₃₀)×0.2/5; and C_{c120}=Cₘ₁₂₀+(Cₘ₁₅+Cₘ₃₀+Cₘ₆₀)×0.2/5.

The Papp for each compound was calculated as follows: Pₐₚₚ=(dCᵣ/dt)×Vᵣ/(A×Cₙ), where dCᵣ/dt is the slope of the cumulative receiver concentration (in μM) vs. time (t); Vᵣ is the volume of the receiver compartment (5 cm³); A is the exposed surface area for permeation (0.6 cm²); and Cₙ is the nominal concentration in the donor compartment (in μM).

The recovery (%) for each reference compound was calculated as follows: Recovery=100×((Vᵣ×C^{final})+(V_{d}^{initial}×C_{d}^{initial})+(V_{d}^{final}×C_{d}^{final}))/(V_{d}×Cₙ); where Vᵣ is the volume of the receiver compartment (5 cm³); V_{d} is the volume of the donor compartment (0.3 cm³); V_{d}^{initial} is the volume sampled from the donor compartment at 0 min (0.1 cm³); Cₙ is the nominal concentration of the donor (in μM); V_{d}^{final} is the volume of the donor compartment at the end of the incubation (0.2 cm³); C^{final} is the cumulative receiver concentration at the end of the incubation (in μM); C_{d}^{final} is the donor concentration at the end of the incubation (in μM); and C_{d}^{initial} is the donor concentration at 0 min (in μM).

The recovery (%) at the final timepoint for histamine was calculated as follows: Recovery=100×((Vᵣ₁₂₀×Cᵣ^{final})+(V_{d}^{initial}×C_{d}^{initial})+(V_{d}¹⁵×C_{d}¹⁵)+(V_{d}³⁰×C_{d}³⁰)+(V_{d}⁶⁰×C_{d}⁶⁰)+(V_{d}^{final}×C_{d}^{final}))/(V_{d}×Cₙ).

The recovery (%) for histamine at each intermediate time point was calculated as follows: Recovery₀=100×(V_{d}×Average C_{d}^{initial})/(V_{d}×Cₙ); Recovery₁₅=100×((Vᵣ₁₅×Average C¹⁵)+(V_{d}^{initial}×Average C_{d}^{initial})+(V_{d}^{f}×Average C¹⁵))/(V_{d}×Cₙ); Recovery₃₀=100×((Vᵣ₃₀×Average C³⁰)+(V_{d}^{initial}×Average C_{d}^{initial})+(V_{d}¹⁵×Average; and Recovery₆₀=100×((Vᵣ₆₀×Average C⁶⁰)+(V_{d}^{initial}×Average C_{d}^{initial})+(V_{d}¹⁵×Average); where Vᵣₓ is the volume of the receiver compartment at “x” min (5 cm³); V_{d} is the volume of the donor compartment (0.2 cm³); V_{d}^{initial} is the volume sampled from the donor compartment at 0 min (0.01 cm³); Cₙ is the nominal concentration of the donor (in μM); C^{final} is the cumulative receiver concentration at the end of the incubation (in μM); C^{x} is the cumulative receiver concentration at “x” min (in μM); C_{d}^{final} is the donor concentration at the end of the incubation (in μM); C_{d}^{initial} is the donor concentration at 0 min (in μM); V_{d}^{x} is the volume sampled from the donor compartment at “x” min (0.01 cm³); V_{d}^{f} is the volume of the donor compartment at the final “x” min (0.19 at 15 minutes, 0.18 at 30 minutes, and 0.17 at 60 minutes in cm³); C_{d}^{x} is the donor concentration at “x” min (in μM); and V_{d}^{final} is the final volume of the donor compartment (0.16 cm³).

The flux was calculated using the following equation: Flux(overall)=dCc/dt×Vr/A; where dCc/dt is the slope of the cumulative receiver concentration (in μM) vs. time (t); Vr is the volume of the receiver compartment (in cm³); t is the duration of the flux period in the linear range (in minutes); and A is the diffusional surface area of the exposed tissue surface (0.6 cm²).

Only samples collected in a linear range (meeting the linearity criterion of r²≥0.9 for cumulative receiver concentration vs. t using the “RSQ” function in Microsoft Office 2016 Excel) were used to calculate dCr/dt values.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

While the subject matter has been particularly shown and described with reference to a preferred embodiment and various alternate embodiments, it will be understood by persons skilled in the relevant art that various changes in form and details can be made therein without departing from the spirit and scope of the present disclosure.

1. A pharmaceutical composition comprising delayed release beads, the delayed release beads comprising:
a solid dispersion of a therapeutically effective dosage of at least one active pharmaceutical ingredient (API), wherein the API comprises histamine, a histamine salt, a histamine derivative, a salt of a histamine derivative, and any combination of the foregoing; and
a delayed release coating comprising a water insoluble polymer and an enteric polymer, wherein the delayed release coating is insoluble at pH equal to or less than about 3.5 and is soluble at pH equal to or greater than about 6.

2. A pharmaceutical composition comprising an enteric tablet, the enteric tablet comprising a compressed tablet having an outer surface and a polymeric coating located on the outer surface, wherein the compressed tablet comprises a therapeutically effective dosage of at least one API, wherein the API comprises histamine, a histamine salt, a histamine derivative, a salt of a histamine derivative, and any combination of the foregoing, and wherein the polymeric coating is insoluble at pH equal to or less than about 3.5 and is soluble at pH equal to or greater than about 6.

3. The composition of claim 1, wherein the composition provides a therapeutically effective plasma concentration of the API of equal to or greater than about 0.2 μmole/L.

4. The composition of claim 1, wherein the composition provides the therapeutically effective plasma concentration for about 0.25 to about 5 hours.

5. The composition of claim 1, wherein the histamine salt comprises a histamine monocation or a histamine polycation.

6. The composition of claim 1, wherein the histamine salt comprises at least one anion selected from acetate, aspartate, citrate, formate, fumarate, halide, malate, nitrate, nitrite, phosphite, phosphate, succinate, sulfate, sulfite, tartrate, and any combination of the foregoing.

7. The composition of claim 1, wherein the histamine salt comprises histamine dihydrochloride.

8. The composition of claim 1, wherein the histamine derivative comprises a C₁₋₆ alkyl histamine.

9. The composition of claim 1, wherein the histamine derivative comprises N-methylhistamine, 1-methylhistamine, 2-methylhistamine, 4-methylhistamine, 5-methylhistamine, alpha-methylhistamine, and any combination of the foregoing.

10. The composition of claim 1, wherein the salt of the histamine derivative comprises a C₁₋₆ alkyl histamine monocation or a C₁₋₆ alkyl histamine polycation.

11. The composition of claim 1, wherein the salt of the histamine derivative comprises at least one anion selected from acetate, aspartate, citrate, formate, fumarate, halide, malate, nitrate, nitrite, phosphite, phosphate, succinate, sulfate, sulfite, tartrate, and any combination of the foregoing.

12. The composition of claim 1, wherein the salt of the histamine derivative comprises N-methylhistamine dihydrochloride, 1-methylhistamine dihydrochloride, 2-methylhistamine dihydrochloride, 4-methylhistamine dihydrochloride, 5-methylhistamine dihydrochloride, alpha-methylhistamine dihydrochloride, and any combination of the foregoing.

13. The composition of claim 1, wherein the solid dispersion comprises at least one a pharmaceutically acceptable solid buffer comprising an organic acid or an alkaline buffer.

14. The composition of claim 13, wherein the organic acid comprises aspartic acid, fumaric acid, acetic acid, formic acid, succinic acid, lactic acid, malic acid, tartaric acid, citric acid, ascorbic acid, nicotinic acid, methanesulfonic acid, ethanesulfonic acid, p-toluensulfonic acid, salicylic acid, naphthalenesulfonic acid, and any combination of the foregoing.

15. The composition of claim 13, wherein the alkaline buffer comprises a cation comprising an alkali metal, an alkaline earth metal and any combination of the foregoing and an anion comprising acetate, citrate, formate, halide, malate, nitrate, nitrite, phosphite, phosphate, succinate, sulfate, sulfite, tartrate, and any combination of the foregoing.

16-22. (canceled)

23. The composition of claim 1, wherein the therapeutically effective dosage is from about 0.1 mg to about 10 mg.

24. A method of preparing the delayed release beads of claim 1, comprising:
dissolving the API and sufficient solubility-enhancing polymer in a pharmaceutically acceptable solvent, thereby forming a solution;
removing the pharmaceutically acceptable solvent from the solution, whereby particles of a solid dispersion are formed;
dissolving the water insoluble polymer and the enteric polymer in a pharmaceutically acceptable coating solvent, thereby forming a delayed release coating solution;
coating the particles of solid dispersion with the delayed release coating solution;
removing the coating solvent, thereby forming delayed release beads comprising a delayed release coating formed on the particles of the solid dispersion.

25. A method for treating a cancer or a tumor, the method comprising:
administering a pharmaceutical composition to a subject having the cancer or the tumor, the pharmaceutical composition comprising delayed release beads, wherein the delayed release beads comprise:
a solid dispersion of a therapeutically effective dosage of at least one active pharmaceutical ingredient (API), wherein the API comprises histamine, a histamine salt, a histamine derivative, a salt of a histamine derivative, and any combination of the foregoing; and
a delayed release coating comprising a water insoluble polymer and an enteric polymer, wherein the delayed release coating is insoluble at pH equal to or less than about 3.5 and is soluble at pH equal to or greater than about 6; and
providing a therapeutically effective plasma concentration of the API of equal to or greater than about 0.2 μmole/L.

26-44. (canceled)

45. A method for treating a cancer comprising contacting a malignant growth or a tumor with a pharmaceutical composition of claim 1.

46. A method for inhibiting replication of a malignant growth or a tumor comprising contacting the growth or the tumor with a pharmaceutical composition of claim 1.

47-50. (canceled)
